SEQUENCE LISTING (23 sequences)

SEQ ID NO: 1 - DNA, 987 bp, Homo sapiens
  source 1..987: mol_type = genomic DNA
  CDS 61..420
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 2 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 3 - DNA, 1611 bp, Homo sapiens
  source 1..1611: mol_type = genomic DNA
  CDS 85..1182
gagaagccaatcagtgtcgtcgcggtcgctgttctaaagtccgcacgcacccaccgggactcagattctccccagacgccgaggatggccgtcatggcgccccgaaccctcctcctgctactctcgggggccctggccctgacccagacctgggcgggctcccactccatgaggtatttcttcacatccgtgtcccggcccggccgcggggagccccgcttcatcgccgtgggctacgtggacgacacgcagttcgtgcggttcgacagcgacgccgcgagccagaagatggagccgcgggcgccgtggatagagcaggaggggccggagtattgggaccaggagacacggaatatgaaggcccactcacagactgaccgagcgaacctggggaccctgcgcggctactacaaccagagcgaggacggttctcacaccatccagataatgtatggctgcgacgtggggccggacgggcgcttcctccgcgggtaccggcaggacgcctacgacggcaaggattacatcgccctgaacgaggacctgcgctcttggaccgcggcggacatggcagctcagatcaccaagcgcaagtgggaggcggtccatgcggcggagcagcggagagtctacctggagggccggtgcgtggacgggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctgtcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtctctcacagcttgtaaagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagagcaccccacccccatgtccaccatgaccctcttcccacgctgacctgtgctccctctccaatcatctttcctgttccagagaggtggggctgaggtgtctccatctctgtctcaacttcatggtgcactgagctgtaacttcttccttccctattaaaattagaacctgagtataaatttactttctcaaattcttgccatgagaggttgatgagttaattaaaggagaagattcctaaaatttgagagacaaaattaatggaacgcatgagaaccttccagagtcca

SEQ ID NO: 4 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 5 - DNA, 1578 bp, Homo sapiens
  source 1..1578: mol_type = genomic DNA
  CDS 55..1143
agttctaaagtccccacgcacccacccggactcagagtctcctcagacgccgagatgctggtcatggcgccccgaaccgtcctcctgctgctctcggcggccctggccctgaccgagacctgggccggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccggaacacacagatctacaaggcccaggcacagactgaccgagagagcctgcggaacctgcgcggctactacaaccagagcgaggccgggtctcacaccctccagagcatgtacggctgcgacgtggggccggacgggcgcctcctccgcgggcatgaccagtacgcctacgacggcaaggattacatcgccctgaacgaggacctgcgctcctggaccgccgcggacacggcggctcagatcacccagcgcaagtgggaggcggcccgtgaggcggagcagcggagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccgtccccatcgtgggcattgttgctggcctggctgtcctagcagttgtggtcatcggagctgtggtcgctgctgtgatgtgtaggaggaagagttcaggtggaaaaggagggagctactctcaggctgcgtgcagcgacagtgcccagggctctgatgtgtctctcacagcttgaaaagcctgagacagctgtcttgtgagggactgagatgcaggatttcttcacgcctcccctttgtgacttcaagagcctctggcatctctttctgcaaaggcacctgaatgtgtctgcgtccctgttagcataatgtgaggaggtggagagacagcccacccttgtgtccactgtgacccctgttcccatgctgacctgtgtttcctccccagtcatctttcttgttccagagaggtggggctggatgtctccatctctgtctcaactttacgtgcactgagctgcaacttcttacttccctactgaaaataagaatctgaatataaatttgttttctcaaatatttgctatgagaggttgatggattaattaaataagtcaattcctggaatttgagagagcaaataaagacctgagaaccttccagaaaaaaaa

SEQ ID NO: 6 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 7 - DNA, 1586 bp, Homo sapiens
  source 1..1586: mol_type = genomic DNA
  CDS 66..1166
tccgcagtcccggttctaaagtccccagtcacccacccggactcacattctccccagaggccgagatgcgggtcatggcgccccgagccctcctcctgctgctctcgggaggcctggccctgaccgagacctgggcctgctcccactccatgaggtatttcgacaccgccgtgtcccggcccggccgcggagagccccgcttcatctcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacaggctgaccgagtgagcctgcggaacctgcgcggctactacaaccagagcgaggacgggtctcacaccctccagaggatgtctggctgcgacctggggcccgacgggcgcctcctccgcgggtatgaccagtccgcctacgacggcaaggattacatcgccctgaacgaggacctgcgctcctggaccgccgcggacaccgcggctcagatcacccagcgcaagttggaggcggcccgtgcggcggagcagctgagagcctacctggagggcacgtgcgtggagtggctccgcagatacctggagaacgggaaggagacgctgcagcgcgcagaacccccaaagacacacgtgacccaccaccccctctctgaccatgaggccaccctgaggtgctgggccctgggcttctaccctgcggagatcacactgacctggcagcgggatggggaggaccagacccaggacaccgagcttgtggagaccaggccagcaggagatggaaccttccagaagtgggcagctgtggtggtgccttctggacaagagcagagatacacgtgccatatgcagcacgaggggctgcaagagcccctcaccctgagctgggagccatcttcccagcccaccatccccatcatgggcatcgttgctggcctggctgtcctggttgtcctagctgtccttggagctgtggtcaccgctatgatgtgtaggaggaagagctcaggtggaaaaggagggagctgctctcaggctgcgtgcagcaacagtgcccagggctctgatgagtctctcatcacttgtaaagcctgagacagctgcctgtgtgggactgagatgcaggatttcttcacacctctcctttgtgacttcaagagcctctggcatctctttctgcaaaggcacctgaatgtgtctgcgttcctgttagcataatgtgaggaggtggagagacagcccacccccgtgtccaccgtgacccctgtccccacactgacctgtgttccctccccgatcatctttcctgttccagagaggtggggctggatgtctccatctctgtctcaaattcatggtgcactgagctgcaacttcttacttccctaatgaagttaagaacctgaatataaatttgtgttctcaaatatttgctatgaagcgttgatggattaattaaataagtcaattcctagaagttgagagagcaaataaagacctgagaaccttccagaa

SEQ ID NO: 8 - protein, 366 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 9 - DNA, 2679 bp, Homo sapiens
  source 1..2679: mol_type = genomic DNA
  CDS 127..1203
gcagactcagttctcattcccaatgggtgtcgggtttctagagaagccaatcagcgtcgccacgactcccgactataaagtccccatccggactcaagaagttctcaggactcagaggctgggatcatggtagatggaaccctccttttactcctctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaaagcctgagacagctgccttgtgtgcgactgagatgcacagctgccttgtgtgcgactgagatgcaggatttcctcacgcctcccctatgtgtcttaggggactctggcttctctttttgcaagggcctctgaatctgtctgtgtccctgttagcacaatgtgaggaggtagagaaacagtccacctctgtgtctaccatgacccccttcctcacactgacctgtgttccttccctgttctcttttctattaaaaataagaacctgggcagagtgcggcagctcatgcctgtaatcccagcacttagggaggccgaggagggcagatcacgaggtcaggagatcgaaaccatcctggctaacacggtgaaaccccgtctctactaaaaaatacaaaaaattagctgggcgcagaggcacgggcctgtagtcccagctactcaggaggcggaggcaggagaatggcgtcaacccgggaggcggaggttgcagtgagccaggattgtgcgactgcactccagcctgggtgacagggtgaaacgccatctcaaaaaataaaaattgaaaaataaaaaaagaacctggatctcaatttaatttttcatattcttgcaatgaaatggacttgaggaagctaagatcatagctagaaatacagataattccacagcacatctctagcaaatttagcctattcctattctctagcctattccttaccacctgtaatcttgaccatataccttggagttgaatattgttttcatactgctgtggtttgaatgttccctccaacactcatgttgagacttaatccctaatgtggcaatactgaaaggtggggcctttgagatgtgattggatcgtaaggctgtgccttcattcatgggttaatggattaatgggttatcacaggaatgggactggtggctttataagaagaggaaaagagaactgagctagcatgcccagcccacagagagcctccactagagtgatgctaagtggaaatgtgaggtgcagctgccacagagggcccccaccagggaaatgtctagtgtctagtggatccaggccacaggagagagtgccttgtggagcgctgggagcaggacctgaccaccaccaggaccccagaactgtggagtcagtggcagcatgcagcgcccccttgggaaagctttaggcaccagcctgcaacccattcgagcagccacgtaggctgcacccagcaaagccacaggcacggggctacctgaggccttgggggcccaatccctgctccagtgtgtccgtgaggcagcacacgaagtcaaaagagattattctcttcccacagataccttttctctcccatgaccctttaacagcatctgcttcattcccctcaccttcccaggctgatctgaggtaaactttgaagtaaaataaaagctgtgtttgagcatcatttgtatttcaaaaaaaaaaaaaaaaaa

SEQ ID NO: 10 - protein, 358 aa, Homo sapiens
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 11 - DNA, 1301 bp, Homo sapiens
  source 1..1301: mol_type = genomic DNA
  CDS 125..1165
tttctcactcccattgggcgtcgcgtttctagagaagccaatcagtgtcgccgcagttcccaggttctaaagtcccacgcaccccgcgggactcatatttttcccagacgcggaggttggggtcatggcgccccgaagcctcctcctgctgctctcaggggccctggccctgaccgatacttgggcgggctcccactccttgaggtatttcagcaccgctgtgtcgcggcccggccgcggggagccccgctacatcgccgtggagtacgtagacgacacgcaattcctgcggttcgacagcgacgccgcgattccgaggatggagccgcgggagccgtgggtggagcaagaggggccgcagtattgggagtggaccacagggtacgccaaggccaacgcacagactgaccgagtggccctgaggaacctgctccgccgctacaaccagagcgaggctgggtctcacaccctccagggaatgaatggctgcgacatggggcccgacggacgcctcctccgcgggtatcaccagcacgcgtacgacggcaaggattacatctccctgaacgaggacctgcgctcctggaccgcggcggacaccgtggctcagatcacccagcgcttctatgaggcagaggaatatgcagaggagttcaggacctacctggagggcgagtgcctggagttgctccgcagatacttggagaatgggaaggagacgctacagcgcgcagatcctccaaaggcacacgttgcccaccaccccatctctgaccatgaggccaccctgaggtgctgggccctgggcttctaccctgcggagatcacgctgacctggcagcgggatggggaggaacagacccaggacacagagcttgtggagaccaggcctgcaggggatggaaccttccagaagtgggccgctgtggtggtgcctcctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtgtgagacagcttccttgtgtgggactgagaagcaagatatcaatgtagcagaattgcacttgtgcctcacgaacatacataaattttaaaaataaagaataaaaatatatctttttatagataaaaaaaaaaaaaaaaaa

SEQ ID NO: 12 - protein, 346 aa, Homo sapiens
MAPRSLLLLLSGALALTDTWAGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPPGEEQRYTCHVQHEGLPQPLILRWEQSPQPTIPIVGIVAGLVVLGAVVTGAVVAAVMWRKKSSDRNRGSYSQAAV

SEQ ID NO: 13 - DNA, 1578 bp, Homo sapiens
  source 1..1578: mol_type = genomic DNA
  CDS 179..1195
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaacttt

SEQ ID NO: 14 - protein, 338 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 15 - DNA, 9232 bp, synthetic construct
  misc_feature 1..9232: note = Vector sequence for human B2M-HLA-A0201 fusion protein
  source 1..9232: mol_type = other DNA
ctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttctagattgtacgggagctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaacctactcatatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgcccaacgtggggctcgaatataagtcgggtttatttgtaaattatccctagggacctccgagcaagcgagcatagcgggaggcatataaaagccaatagacaatggctagcaggaagtaatgttgaagaatatgaacttgatgttgaagctctggttgtaattttaagagatagaaatataccaagaaatcctttacatggagaagttataggtcttcgccttactgaaggatggtggggacaaattgagagatttcagatggtacgttgatctaaggctatggatttggccatgggacaagaaatattagtttatagtcccattgtatctatgactaaaatacaaaaaactccactaccagaaagaaaagctttacccattagatggataacatggatgacttatttagaagatccaagaatccaatttcattatgataaaaccttaccagaacttaagcatattccagatgtatatacatctagtcagtctcctgttaaacatccttctcaatatgaaggagtgttttatactgatggctcggccatcaaaagtcctgatcctacaaaaagcaataatgctggcatgggaatagtacatgccacatacaaacctgaatatcaagttttgaatcaatggtcaataccactaggtaatcatactgctcagatggctgaaatagctgcagttgaatttgcctgtaaaaaagctttaaaaatacctggtcctgtattagttataactgatagtttctatgtagcagaaagtgctaataaagaattaccatactggaaatctaatgggtttgttaataataagaaaaagcctcttaaacatatctccaaatggaaatctattgctgagtgtttatctatgaaaccagacattactattcaacatgaaaaaggcatcagcctacaaataccagtattcatactgaaaggcaatgccctagcagataagcttgccacccaaggaagttatgtggttaattgtaataccaaaaaaccaaacctggatgcagagttggatcaattattacagggtcattatataaaaggatatcccaaacaatatacatattttttagaagatggcaaagtaaaagtttccagacctgaaggggttaaaattattccccctcagtcagacagacaaaaaattgtgcttcaagcccacaatttggctcacaccggacgtgaagccactcttttaaaaattgccaacctttattggtggccaaatatgagaaaggatgtggttaaacaactaggacgctgtcaacagtgtttaatcacaaatgcttccaacaaagcctctggtcctattctaagaccagataggcctcaaaaaccttttgataaattctttattgactatattggacctttgccaccttcacagggatacctatatgtattagtagttgttgatggaatgacaggattcacttggttataccccactaaggctccttctactagcgcaactgttaaatctctcaatgtactcactagtattgcaattccaaaggtgattcactctgatcaaggtgcagcattcacttcttcaacctttgctgaatgggcaaaggaaagaggtatacatttggaattcagtactccttatcacccccaaagtggtagtaaggtggaaaggaaaaatagtgatataaaacgacttttaactaaactgctagtaggaagacccacaaagtggtatgacctattgcctgttgtacaacttgctttaaacaacacctatagccctgtattaaaatatactccacatcaactcttatttggtatagattcaaatactccatttgcaaatcaagatacacttgacttgaccagagaagaagaactttctcttttacaggaaattcgtacttctttataccatccatccacccctccagcctcctctcgttcctggtctcctgttgttggccaattggtccaggagagggtggctaggcctgcttctttgagacctcgttggcataaaccgtctactgtacttaaggtgttgaatccaaggactgttgttattttggaccatcttggcaacaacagaactgtaagtatagataatttaaaacctacttctcatcagaatggcaccaccaatgacactgcaacaatggatcatttggaaaaaaatgaataaagcgcatgaggcacttcaaaatacaacaactgtgactgaacagcagaaggaacaaattatactggacattcaaaatgaagaagtacaaccaactaggagagataaatttagatatctgctttatacttgttgtgctactagctcaagagtattggcctggatgtttttagtttgtatattgttaatcattgttttggtttcatgctttgtgactatatccagaatacaatggaataaggatattcaggtattaggacctgtaatagactggaatgttactcaaagagctgtttatcaacccttacagactagaaggattgcacgttcccttagaatgcagcatcctgttccaaaatatgtggaggtaaatatgactagtattccacaaggtgtatactatgaaccccatccggcctcgacggtatcgatggtaccggtatcgataagcttgataacctcgagagatctaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccacttctactcctcccctagtcaggaagtttcccccagcaagctcgcgtcgtgcaggacgtgacaaatggaagtagcacgtctcactagtctcgtgcagatggacagcaccgctgagcaatggaagcgggtaggcctttggggcagcggccaatagcagctttgttccttcgctttctgggctcagaggctgggaaggggtgggtccgggggcgggctcaggggcgggctcaggggcgggcgggcgcccgaaggtcctcccgaggcccggcattctgcacgcttcaaaagcgcacgtctgccgcgctgttctcctcttcctcatctccgggcctttcgacctgcagcccgggggatcgatcctaggtgatttaaatccaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacggatccatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgcggccgcgccgccaccatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggaggcggaagcgggggcggcggatccggcggaggcggaagcggcggcgggggaagcggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtgagcggccgcgactctagatcaggcgcgccgttaccaagcagctatggaagcttatggacctcagagaggaagtaacgaggagagggtgtggtggaatgtcactagaaaccagggaaaacaaggaggagagtattacagggaaggaggtgaagaacctcattacccaaatactcctgctcctcatagacgtacctgggatgagagacacaaggttcttaaattgtcctcattcgctactccctctgacatccaacgctgggctactaactctagattgtacgggagctctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaacctactcatatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgataccgtcgaccgttcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 16 - protein, 480 aa, synthetic construct
  REGION 1..480: note = HLA-bBA0201 (human B2M-HLA-A0201 fusion peptide)
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 17 - DNA, 9224 bp, synthetic construct
  misc_feature 1..9224: note = Vector sequence for HLA-gBE
  source 1..9224: mol_type = other DNA
ctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttctagattgtacgggagctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaacctactcatatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgcccaacgtggggctcgaatataagtcgggtttatttgtaaattatccctagggacctccgagcatagcgggaggcatataaaagccaatagacaatggctagcaggaagtaatgttgaagaatatgaacttgatgttgaagctctggttgtaattttaagagatagaaatataccaagaaatcctttacatggagaagttataggtcttcgccttactgaaggatggtggggacaaattgagagatttcagatggtacgttgatctaaggctatggatttggccatgggacaagaaatattagtttatagtcccattgtatctatgactaaaatacaaaaaactccactaccagaaagaaaagctttacccattagatggataacatggatgacttatttagaagatccaagaatccaatttcattatgataaaaccttaccagaacttaagcatattccagatgtatatacatctagtcagtctcctgttaaacatccttctcaatatgaaggagtgttttatactgatggctcggccatcaaaagtcctgatcctacaaaaagcaataatgctggcatgggaatagtacatgccacatacaaacctgaatatcaagttttgaatcaatggtcaataccactaggtaatcatactgctcagatggctgaaatagctgcagttgaatttgcctgtaaaaaagctttaaaaatacctggtcctgtattagttataactgatagtttctatgtagcagaaagtgctaataaagaattaccatactggaaatctaatgggtttgttaataataagaaaaagcctcttaaacatatctccaaatggaaatctattgctgagtgtttatctatgaaaccagacattactattcaacatgaaaaaggcatcagcctacaaataccagtattcatactgaaaggcaatgccctagcagataagcttgccacccaaggaagttatgtggttaattgtaataccaaaaaaccaaacctggatgcagagttggatcaattattacagggtcattatataaaaggatatcccaaacaatatacatattttttagaagatggcaaagtaaaagtttccagacctgaaggggttaaaattattccccctcagtcagacagacaaaaaattgtgcttcaagcccacaatttggctcacaccggacgtgaagccactcttttaaaaattgccaacctttattggtggccaaatatgagaaaggatgtggttaaacaactaggacgctgtcaacagtgtttaatcacaaatgcttccaacaaagcctctggtcctattctaagaccagataggcctcaaaaaccttttgataaattctttattgactatattggacctttgccaccttcacagggatacctatatgtattagtagttgttgatggaatgacaggattcacttggttataccccactaaggctccttctactagcgcaactgttaaatctctcaatgtactcactagtattgcaattccaaaggtgattcactctgatcaaggtgcagcattcacttcttcaacctttgctgaatgggcaaaggaaagaggtatacatttggaattcagtactccttatcacccccaaagtggtagtaaggtggaaaggaaaaatagtgatataaaacgacttttaactaaactgctagtaggaagacccacaaagtggtatgacctattgcctgttgtacaacttgctttaaacaacacctatagccctgtattaaaatatactccacatcaactcttatttggtatagattcaaatactccatttgcaaatcaagatacacttgacttgaccagagaagaagaactttctcttttacaggaaattcgtacttctttataccatccatccacccctccagcctcctctcgttcctggtctcctgttgttggccaattggtccaggagagggtggctaggcctgcttctttgagacctcgttggcataaaccgtctactgtacttaaggtgttgaatccaaggactgttgttattttggaccatcttggcaacaacagaactgtaagtatagataatttaaaacctacttctcatcagaatggcaccaccaatgacactgcaacaatggatcatttggaaaaaaatgaataaagcgcatgaggcacttcaaaatacaacaactgtgactgaacagcagaaggaacaaattatactggacattcaaaatgaagaagtacaaccaactaggagagataaatttagatatctgctttatacttgttgtgctactagctcaagagtattggcctggatgtttttagtttgtatattgttaatcattgttttggtttcatgctttgtgactatatccagaatacaatggaataaggatattcaggtattaggacctgtaatagactggaatgttactcaaagagctgtttatcaacccttacagactagaaggattgcacgttcccttagaatgcagcatcctgttccaaaatatgtggaggtaaatatgactagtattccacaaggtgtatactatgaaccccatccggcctcgacggtatcgatggtaccggtatcgataagcttgataacctcgagagatctaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccacttctactcctcccctagtcaggaagtttcccccagcaagctcgcgtcgtgcaggacgtgacaaatggaagtagcacgtctcactagtctcgtgcagatggacagcaccgctgagcaatggaagcgggtaggcctttggggcagcggccaatagcagctttgttccttcgctttctgggctcagaggctgggaaggggtgggtccgggggcgggctcaggggcgggctcaggggcgggcgggcgcccgaaggtcctcccgaggcccggcattctgcacgcttcaaaagcgcacgtctgccgcgctgttctcctcttcctcatctccgggcctttcgacctgcagcccgggggatcgatcctaggtgatttaaatccaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacggatccatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgcggccgcgttaaccatggtggtcatggcccctcgaactctgttcctgctgctgagtggggctctgaccctgacagagtccggaatccagcggacccccaagattcaggtgtacagcagacaccctgcagagaacggcaaatccaacttcctgaattgctatgtgtctgggtttcatcccagtgacatcgaagtcgatctgctgaagaatggcgagagaattgaaaaagtcgagcactctgacctgagcttctccaaggattggtccttttacctgctgtactataccgagtttactccaaccgaaaaagacgagtatgcctgtagggtgaaccatgtcacactgagccagcccaagatcgtgaaatgggaccgcgatatgggcgggggaggctcagggggaggcgggagcggaggcgggggctccggcgggggaggatccggttctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatctagagcggccgcgactctagatcaggcgcgccgttaccaagcagctatggaagcttatggacctcagagaggaagtaacgaggagagggtgtggtggaatgtcactagaaaccagggaaaacaaggaggagagtattacagggaaggaggtgaagaacctcattacccaaatactcctgctcctcatagacgtacctgggatgagagacacaaggttcttaaattgtcctcattcgctactccctctgacatccaacgctggactactaactctagattgtacgggaggctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaaccaactcctatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgataccgtcgaccgttcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 18 - protein, 476 aa, synthetic construct
  REGION 1..476: note = HLA-gBE
MAPRTLFLLLSGALTLTESGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 19 - DNA, 9287 bp, synthetic construct
  misc_feature 1..9287: note = Vector sequence for HLA-bGBE
  source 1..9287: mol_type = other DNA
ctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttctagattgtacgggagctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaacctactcatatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgcccaacgtggggctcgaatataagtcgggtttatttgtaaattatccctagggacctccgagcatagcgggaggcatataaaagccaatagacaatggctagcaggaagtaatgttgaagaatatgaacttgatgttgaagctctggttgtaattttaagagatagaaatataccaagaaatcctttacatggagaagttataggtcttcgccttactgaaggatggtggggacaaattgagagatttcagatggtacgttgatctaaggctatggatttggccatgggacaagaaatattagtttatagtcccattgtatctatgactaaaatacaaaaaactccactaccagaaagaaaagctttacccattagatggataacatggatgacttatttagaagatccaagaatccaatttcattatgataaaaccttaccagaacttaagcatattccagatgtatatacatctagtcagtctcctgttaaacatccttctcaatatgaaggagtgttttatactgatggctcggccatcaaaagtcctgatcctacaaaaagcaataatgctggcatgggaatagtacatgccacatacaaacctgaatatcaagttttgaatcaatggtcaataccactaggtaatcatactgctcagatggctgaaatagctgcagttgaatttgcctgtaaaaaagctttaaaaatacctggtcctgtattagttataactgatagtttctatgtagcagaaagtgctaataaagaattaccatactggaaatctaatgggtttgttaataataagaaaaagcctcttaaacatatctccaaatggaaatctattgctgagtgtttatctatgaaaccagacattactattcaacatgaaaaaggcatcagcctacaaataccagtattcatactgaaaggcaatgccctagcagataagcttgccacccaaggaagttatgtggttaattgtaataccaaaaaaccaaacctggatgcagagttggatcaattattacagggtcattatataaaaggatatcccaaacaatatacatattttttagaagatggcaaagtaaaagtttccagacctgaaggggttaaaattattccccctcagtcagacagacaaaaaattgtgcttcaagcccacaatttggctcacaccggacgtgaagccactcttttaaaaattgccaacctttattggtggccaaatatgagaaaggatgtggttaaacaactaggacgctgtcaacagtgtttaatcacaaatgcttccaacaaagcctctggtcctattctaagaccagataggcctcaaaaaccttttgataaattctttattgactatattggacctttgccaccttcacagggatacctatatgtattagtagttgttgatggaatgacaggattcacttggttataccccactaaggctccttctactagcgcaactgttaaatctctcaatgtactcactagtattgcaattccaaaggtgattcactctgatcaaggtgcagcattcacttcttcaacctttgctgaatgggcaaaggaaagaggtatacatttggaattcagtactccttatcacccccaaagtggtagtaaggtggaaaggaaaaatagtgatataaaacgacttttaactaaactgctagtaggaagacccacaaagtggtatgacctattgcctgttgtacaacttgctttaaacaacacctatagccctgtattaaaatatactccacatcaactcttatttggtatagattcaaatactccatttgcaaatcaagatacacttgacttgaccagagaagaagaactttctcttttacaggaaattcgtacttctttataccatccatccacccctccagcctcctctcgttcctggtctcctgttgttggccaattggtccaggagagggtggctaggcctgcttctttgagacctcgttggcataaaccgtctactgtacttaaggtgttgaatccaaggactgttgttattttggaccatcttggcaacaacagaactgtaagtatagataatttaaaacctacttctcatcagaatggcaccaccaatgacactgcaacaatggatcatttggaaaaaaatgaataaagcgcatgaggcacttcaaaatacaacaactgtgactgaacagcagaaggaacaaattatactggacattcaaaatgaagaagtacaaccaactaggagagataaatttagatatctgctttatacttgttgtgctactagctcaagagtattggcctggatgtttttagtttgtatattgttaatcattgttttggtttcatgctttgtgactatatccagaatacaatggaataaggatattcaggtattaggacctgtaatagactggaatgttactcaaagagctgtttatcaacccttacagactagaaggattgcacgttcccttagaatgcagcatcctgttccaaaatatgtggaggtaaatatgactagtattccacaaggtgtatactatgaaccccatccggcctcgacggtatcgatggtaccggtatcgataagcttgataacctcgagagatctaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccacttctactcctcccctagtcaggaagtttcccccagcaagctcgcgtcgtgcaggacgtgacaaatggaagtagcacgtctcactagtctcgtgcagatggacagcaccgctgagcaatggaagcgggtaggcctttggggcagcggccaatagcagctttgttccttcgctttctgggctcagaggctgggaaggggtgggtccgggggcgggctcaggggcgggctcaggggcgggcgggcgcccgaaggtcctcccgaggcccggcattctgcacgcttcaaaagcgcacgtctgccgcgctgttctcctcttcctcatctccgggcctttcgacctgcagcccgggggatcgatcctaggtgatttaaatccaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacggatccatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgcggccgcgttaaccatgagccgatccgtggcactggctgtcctggctctgctgtctctgagtggcctggaagcagtgatggcccctagaacactgttcctgggcggaggcggctccggaggaggagggtctggaggcgggggaagtatccagcggactcccaagattcaggtctacagcagacaccctgccgaaaacgggaaatccaacttcctgaattgctatgtgtcaggctttcatcccagcgacatcgaggtcgatctgctgaagaatggcgagcggattgaaaaagtggagcactctgacctgtcattcagcaaggattggagcttttacctgctgtactatactgagtttaccccaacagaaaaagacgagtatgcctgtagggtgaaccatgtcaccctgagtcagcccaagatcgtgaaatgggaccgcgatatgggcgggggaggctccgggggaggcggctccggcggcgggggaagtggcgggggaggatccggttctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatctagagcggccgcgactctagatcaggcgcgccgttaccaagcagctatggaagcttatggacctcagagaggaagtaacgaggagagggtgtggtggaatgtcactagaaaccagggaaaacaaggaggagagtattacagggaaggaggtgaagaacctcattacccaaatactcctgctcctcatagacgtacctgggatgagagacacaaggttcttaaattgtcctcattcgctactccctctgacatccaacgctggactactaactctagattgtacgggaggctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaaccaactcctatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgataccgtcgaccgttcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 20 - protein, 500 aa, synthetic construct
  REGION 1..500: note = HLA-bGBE
MSRSVALAVLALLSLSGLEAVMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 21 - DNA, 7159 bp, synthetic construct
  misc_feature 1..7159: note = Vector sequence for pA2-B2METKNpA
  source 1..7159: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgatgcagtccaaactctcactaaaattgccgagccctttgtcttccagtgtctaaaatattaatgtcaatggaatcaggccagagtttgaattctagtctcttagcctttgtttcccctgtccataaaatgaatgggggtaattctttcctcctacagtttatttatatattcactaattcattcattcatccatccattcgttcattcggtttactgagtacctactatgtgccagcccctgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatctagataacttcgtataatgtatgctatacgaagttatggatccatcgattctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttccaccatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagggtctctagcataacttcgtataatgtatgctatacgaagttataataaaagatccttattttcattggatctgtgtgttggttttttgtgtgggatccgtcgataccgtcgactgtctcgctccgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagcctaaccagggcttttgcgggagcgcatgccttttggctgtaattcgtgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 22 - DNA, 7199 bp, synthetic construct
  misc_feature 1..7199: note = Vector sequence for pA2-B2MEHyTKpA
  source 1..7199: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatctagataacttcgtataatgtatgctatacgaagttatggatccatcgattctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtcgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgaggtctctagcataacttcgtataatgtatgctatacgaagttataataaaagatccttattttcattggatctgtgtgttggttttttgtgtgggatccgtcgataccgtcgactgtctcgctccgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagcctaaccagggcttttgcgggagcgcatgccttttggctgtaattcgtgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 23 - protein, 29 aa, synthetic construct
  REGION 1..29: note = HLA-G peptide
MAPRTLFLGGGGSGGGGSGGGGSIQRTPK

(19) United States
(12) Patent Application Publication
(10) Pub. No.: US 2024/0207378 A1
(43) Pub. Date: Jun. 27, 2024

(54) BETA-2 MICROGLOBULIN-DEFICIENT CELLS

(71) Applicant: University of Washington through its Center for Commercialization, Seattle, WA (US)

(72) Inventors: David W. Russell, Seattle, WA (US); Roli K. Hirata, Seattle, WA (US)

(21) Appl. No.: 18/485,536

(22) Filed: Oct. 12, 2023

Related U.S. Application Data
(63) Continuation of application No. 16/507,589, filed on Jul. 10, 2019, now Pat. No. 11,813,318.
Application No. 16/507,589 is a continuation of application No. 14/111,837, filed on Dec. 18, 2013, now abandoned.
(60) Provisional application No. 61/477,474, filed on Apr. 20, 2011.

(51) Int. Cl. A61K 39/00; A61K 35/12; A61K 35/28; C07K 14/74

(52) U.S. Cl. CPC A61K 39/0005; A61K 35/28; C07K 14/70539; A61K 35/12; A61K 2039/515; C07K 2319/00; C12N 2740/17043; C12N 2750/14143; C12N 2800/30

(57) ABSTRACT

The invention provides isolated primate cells preferably human cells that comprise a genetically engineered disruption in a beta-2 microglobulin (B2M) gene, which results in deficiency in MHC class I expression and function. Also provided are the method of using the cells for transplantation and treating a disease condition.

This application is a Continuation of U.S. patent application Ser. No. 16/507,589, filed Jul. 10, 2019, which is a Continuation of U.S. patent application Ser. No. 14/111,837, filed Dec. 18, 2013, which is U.S. national phase of International Application No. PCT/US2012/034051, filed on Apr. 18, 2012, which claims priority to U.S. Provisional Application No. 61/477,474, filed Apr. 20, 2011, all of which are incorporated by reference herein in their entirety.

This invention was made with government support under Grant nos. R01 GM086497 and R01 DK055759 awarded by the National Institutes of Health. The government has certain rights in the invention.

A computer readable form of the Sequence Listing is filed with this application by electronic submission and is incorporated into this application by reference in its entirety. The Sequence Listing is contained in the file created on Apr. 24, 2023, having the file name “11-442-PCT.xml” and is 82,415 bytes in size.

### BACKGROUND OF THE INVENTION

Human pluripotent stem cells have the potential to treat diseases affecting almost every organ system. However, the clinical use of human pluripotent stem cells and their derivatives has a major limitation—rejection of transplanted cells by the recipient due to differences in the major histocompatibility complex.

The major histocompatibility complex (MHC) is a cell surface multi-component molecule found in all vertebrates that mediates interactions of leukocytes with other leukocytes or other cells. The MHC gene family is divided into three groups: class I, class II and class III. In humans, MHC is referred to as human leukocyte antigen (HLA). The HLA class I (HLA-I) protein is expressed on all nucleated cells and consists of an HLA class I heavy chain (or α chain) and β-2 microglobulin (B2M). HLA class I protein presents peptides on the cell surface to CD8+ cytotoxic T cells. Six HLA class I α chains have been identified to date, including three classical (HLA-A, HLA-B and HLA-C) and three non-classical (HLA-E, HLA-F and HLA-G) α chains. The specificity for peptide binding on the HLA class I molecule peptide binding cleft is determined by the α chain. Recognition by CD8+ T cells of the peptides presented by the HLA class I molecule mediates cellular immunity.

The HLA class I protein itself from an allogeneic source constitutes a foreign antigen in the context of transplantation. The recognition of non-self HLA class I protein is a major hurdle in using pluripotent cells for transplantation or replacement therapies. The first two clinical trials of human embryonic stem cells (ESCs) have been conducted that delivered ESCs to immune-privileged sites (such as spinal cord and eye) where allogeneic cells might survive. However, even these immune-privileged sites can eventually reject allogeneic cells, and most potential clinical applications do not involve immune-privileged sites. Alternatively, HLA-matched or partially matched cells from HLA-typed stem cell banks or pluripotent stem cell (iPSC) lines derived from each patient can be developed for transplantation. However, the development of individually matched cell line requires significant costs, months of cell culture, highly trained personnel, and extensive validation of the final product, all of which must be done with the approval of regulatory agencies. Furthermore, each cell line will likely behave somewhat differently in gene expression patterns, culture characteristics, differentiation potentials, and genetic variations.

Thus, although individualized stem cell preparations or HLA-diverse stem cell banks may address the current problem of transplantation, they require that multiple cell lines be characterized, differentiated into therapeutic cell products, and approved for human administration. This time-consuming, technically difficult, and expensive process is a major factor preventing stem cell-based therapies from entering clinical trials. Thus, there exists a need for a more effective and less expensive cell-based therapies that are not impeded by rejection.

### SUMMARY OF THE INVENTION

In accordance with the present invention, in one aspect the invention provides an isolated primate cell comprising a genetically engineered disruption in a beta-2 microglobulin (B2M) gene. In certain particular embodiments, the cell comprises genetically engineered disruptions of all copies of the B2M gene.

In certain other embodiments, the cell further comprises one or more recombinant immunomodulatory genes. Suitable immunomodulatory genes include without limitation a gene encoding a viral protein that inhibits antigen presentation, a microRNA gene, and a gene that encodes a single chain (SC) fusion human leukocyte antigen (HLA) class I protein as described below. In certain preferred embodiments, the primate cell is a human cell.

In certain preferred embodiments, the one or more immunomodulatory genes comprise a polynucleotide capable of encoding a single chain fusion HLA class I protein. In certain particular embodiments, the single chain fusion HLA class I protein comprises at least a portion of B2M covalently linked to at least a portion of an HLA class I α chain selected from the group consisting of HLA-A, HLA-B, HLA-C, HLA-E, HLA-F and HLA-G. In certain preferred embodiments, the single chain fusion HLA class I protein comprises at least a portion of B2M and at least a portion of an HLA class I α chain selected from the group consisting of HLA-C, HLA-E and HLA-G. In certain other preferred embodiments, the single chain fusion HLA class I protein comprises at least a portion of B2M and at least a portion of an HLA class I a chain selected from the group consisting of HLA-A, HLA-E and HLA-G. In certain particular embodiments, the single chain fusion HLA class I protein comprises at least a portion of B2M and at least a portion of HLA-A0201 (e.g., SEQ ID NO:16). In certain other particular embodiments, the single chain fusion HLA class I protein comprises at least a portion of B2M and at least a portion of HLA-E (e.g., SEQ ID NOs: 18 and 20).

In yet other particular embodiments, the cell has a normal karyotype. In certain other particular embodiments, the cell is a non-transformed cell. In particular, the cell can be a stem cell selected from the group consisting of a hematopoietic stem cell, an embryonic stem cell, an induced pluripotent stem cell, a liver stem cell, a neural stem cell, a pancreatic stem cell and a mesenchymal stem cell. In certain further embodiments, the cell further comprises one or more recombinant genes capable of encoding a suicide gene product. In certain particular embodiments, the suicide gene product comprises a protein selected from the group consisting of thymidine kinase and an apoptotic signaling protein.

In certain preferred embodiments, the stem cell is a pluripotent stem cell that expresses a single chain fusion HLA class I protein comprising at least a portion of B2M and at least a portion of an HLA class I α chain selected from the group consisting of HLA-A, HLA-B, HLA-C, HLA-E, HLA-F and HLA-G. In certain particular embodiments, the single chain fusion HLA class I protein comprises at least a portion of B2M and at least a portion of HLA-A0201.

In certain other particular embodiments, the stem cell is a differentiated cell. In certain embodiments, the differentiated cell is selected from the group consisting of a dendritic cell, a pancreatic islet cell, a liver cell, a muscle cell, a keratinocyte, a neuronal cell, a hematopoietic cell, a lymphocyte, a red blood cell, a platelet, a skeletal muscle cell, an ocular cell, a mesenchymal cell, a fibroblast, a lung cell, a GI tract cell, a vascular cell, en endocrine cell, an adipocyte and a cardiomyocyte. In certain preferred embodiments, the differentiated cell is a human cell expressing a single chain fusion HLA class I protein comprising at least a portion of B2M and at least a portion of an HLA class I α chain selected from the group consisting of HLA-A, HLA-B, HLA-C, HLA-E, HLA-F and HLA-G. In certain particular embodiments, the single chain fusion HLA class I protein comprises at least a portion of B2M and at least a portion of HLA-A0201.

In certain other embodiments, the cell further expresses a target peptide antigen that is presented by the single chain fusion HLA class I protein on the cell surface. In certain particular embodiments, the target peptide antigen is covalently linked to the single chain fusion HLA class I protein. In certain preferred embodiments, the target peptide antigen is derived from a protein of a pathogen or a cancer cell. Thus, in a related aspect, the invention provides a vaccine comprising the B2M−/− cells of the invention, wherein the vaccine is capable of eliciting in a primate an immune response specific for the target peptide antigen. In certain particular embodiments, the vaccine comprises a cell of the invention that is a differentiated dendritic cell. In certain other embodiments, the cell is a human cell of the invention, wherein the cell expresses a cytokine that further enhances the immune response. In certain preferred embodiments, the cytokine is IL2. In certain other preferred embodiments, the cytokine is IFN-γ. In certain embodiments, the immune response comprises a humoral immune response; while in other embodiments, the immune response comprises a cellular immune response. In a further related aspect, the invention provides a kit comprising a vaccine that comprises the isolated cells of the invention and an immune adjuvant. In certain embodiments, the cell is a human cell.

In yet another aspect, the invention provides a method of transplantation in a patient in need thereof comprising the step of administering to the patient an effective amount of the isolated cell of the invention. In certain embodiments, the patient is immune competent. In certain particular embodiments, the patient is a primate and preferably a human. In certain preferred embodiments, the patient is a human and the cell is a human cell. In further embodiments the cell is a stem cell or a differentiated cell, optionally expressing a single chain fusion HLA class I protein.

In yet a further aspect, the invention provides a method of treating a disease condition in a patient in need thereof comprising the step of administering to the patient an effective amount of the B2M−/− cells of the invention, wherein the disease condition includes without limitation an endocrine disorder, diabetes, an autoimmune disease, cancer, infection, anemia, a platelet disorder, immunodeficiency, cytopenia, myocardial infarction, heart failure, liver failure, skeletal or joint condition, a neurological condition, stroke, paralysis, blindness or another visual disorder, muscular dystrophy, osteogenesis imperfecta, pulmonary disease, skin condition, or burns. In certain embodiments, the patient is immune competent. In certain particular embodiments, the patient is a primate and preferably a human. In certain preferred embodiments, the patient is a human and the cell is a human cell. In further embodiments the cell is a stem cell or a differentiated cell, optionally expressing a single chain fusion HLA class I protein. In certain particular embodiments, the disease condition is diabetes and the cell is a differentiated pancreatic islet cell. In further embodiments the differentiated pancreatic islet cell expresses a single chain fusion HLA class I protein.

In another aspect, the invention provides a kit comprising the isolated primate cells, preferably human cells, of the invention. In certain embodiments, the kit is for use in transplantation or for use in treating a disease condition. In certain other embodiments, the kit comprises an implant comprising the isolated primate cells, preferably human cells, of the invention.

Specific embodiments of the present invention will become evident from the following more detailed description of certain preferred embodiments and the claims.

### BRIEF DESCRIPTION OF THE DRAWINGS

FIG. 1A-1C: Creation of B2M−/− ESCs. FIG. 1A is an illustration of the AAV B2M targeting vectors with exons shown in large boxes. FIG. 1B presents Southern blots showing the results of gene targeting and Cre-mediated transgene excision (Cre-out). FIG. 1C presents results of flow cytometry showing a lack of HLA class I expression after gene targeting (with isotype controls).

FIG. 2: Tissue sections of teratoma developed from B2M−/− Cre-out human ESCs transplanted in immune-deficient mice. The sections were stained with DAPI, hematoxylin and eosin, or lineage-specific markers MAP-2 (microtubule associated protein-2) for ectoderm, α-SAM (α-smooth muscle actin) for mesoderm or FoxA2 (forkhead box protein A2) for endoderm. Scale bar=100 microns.

FIG. 3A-3D: Single chain fusion HLA class I constructs. FIG. 3A shows the foamy viral vector design for expressing single chain fusion HLA class I proteins. FIG. 3B illustrates the linear protein structure of single chain fusion HLA class I proteins. Sequences for exemplary single chain fusion HLA class I proteins are provided for HLA-bGBE (SEQ ID NOs: 19 and 20, DNA and protein sequences, respectively), HLA-gBE (SEQ ID NOs: 17 and 18), HLA-bBA0201 (SEQ ID NOs:15 and 16), and (G4S)₃ (SEQ ID NO: 24). FIG. 3C presents results of flow cytometry showing single chain fusion HLA-E expression in B2M−/− ESCs (isotype controls). FIG. 3D presents results of flow cytometry showing single chain fusion HLA-A0201 expression in B2M−/− ESCs (isotype controls).

FIG. 4: outlines the experimental design for each differentiated cell type.

FIG. 5: shows differentiation of keratinocytes from B2M−/− ESCs.

### DETAILED DESCRIPTION OF THE INVENTION

All publications, patents and patent applications cited herein are hereby expressly incorporated by reference for all purposes.

Within this application, unless otherwise stated, the techniques utilized may be found in any of several well-known references such as: Molecular Cloning: A Laboratory Manual (Sambrook, et al., 1989, Cold Spring Harbor Laboratory Press) and PCR Protocols: A Guide to Methods and Applications (Innis, et al. 1990. Academic Press, San Diego, CA).

As used herein, the singular forms “a”, “an” and “the” include plural referents unless the context clearly dictates otherwise. For example, reference to “an isolated cell” means one or more isolated cells.

All embodiments disclosed herein can be combined unless the context clearly dictates otherwise.

In one aspect, the invention provides B2M deficient cells. In particular, the invention provides isolated primate cells, preferably human cells, comprising a genetically engineered disruption in a B2M gene. In certain preferred embodiments, the cell is a human cell comprising a genetically engineered disruption in the B2M gene. In a related aspect, the cell comprises genetically engineered disruptions of all copies of the B2M gene. In certain embodiments, the genetic disruptions in the B2M gene result in defective or no expression of the B2M protein. Since B2M is a common component of all HLA class I proteins, the disruptions preclude the expression of all natural HLA class I proteins on the cell surface. The B2M coding sequence is shown in SEQ ID NO: 1 (GenBank Accession Number NM_004048) and the B2M protein sequence is shown in SEQ ID NO:2. There may be many single nucleotide polymorphisms (SNPs) in the gene; as will be understood by those of skill in the art, the human cells and methods of the invention are applicable to any such B2M gene and SNPs.

The cells of these embodiments of the invention can be used, for example, as donor cells for transplantation in a recipient in need thereof. B2M deficient cells encompass cells that comprise a B2M−/− genetic background (referred to as B2M−/− cells). The term “B2M−/− cells” refers to primate cells, preferably human cells, that comprise genetically engineered disruptions in all copies of the B2M gene. The B2M−/− cells can serve as “universal donor cells” in that they are immunologically compatible to all or a significant percentage of recipients in a population. As used herein, a recipient or patient refers to a primate, and preferably a human. In certain particular embodiments, the cell is a human cell and the patient is a human.

The cells of the invention can be engineered to disrupt the B2M gene such that no functional endogenous B2M protein is produced from the disrupted genetic loci. In certain embodiments, the disruption results in expression of non-functional B2M proteins, including but not limited to truncations, deletions, point mutations and insertions. In other embodiments, the disruption results in no protein expression from the B2M gene.

Cells deficient in B2M expression are unable to express HLA class I proteins on the cell surface. HLA class I-deficiency provides further benefits; for example, cells without HLA class I expression cannot present auto-antigens that would otherwise prevent successful cell therapies for autoimmune diseases such as diabetes and rheumatoid arthritis. Similarly, therapeutic gene products introduced by the inventive cell therapies (e.g., dystrophin) that are missing in patients with certain genetic diseases (e.g., muscular dystrophy) will not be presented and recognized by the immune system as neo-antigens in replacement therapies.

Any suitable technique for disrupting one, two or all copies of the B2M gene can be used; exemplary techniques are disclosed throughout the application and are within the level of skill in the art based on the teachings herein and the teachings known in the art. Exemplary other techniques can be found, for example, in U.S. Patent Application Publication Number US2008/0219956, published Sep. 11, 2008, and incorporated by reference herein in its entirety. These techniques may optionally include steps to remove non-human DNA sequences from the cells after B2M gene disruption.

An exemplary embodiment of this method is as disclosed throughout the application, using an adeno-associated virus gene targeting vector, optionally including removing the transgene used for targeting via techniques such as those described below, or by removing the transgene used for targeting by Cre-mediated loxP recombination, or other suitable recombination techniques. See Khan et al. 2011, Protocol, 6:482-501, which is incorporated by reference in its entirety. Exemplary targeting vectors and exemplary vector diagrams are also disclosed herein. It is within the level of those of skill in the art, based on the teachings herein and known in the art, to utilize a variety of techniques for making the B2M−/− cells, preferably human cells, of the invention.

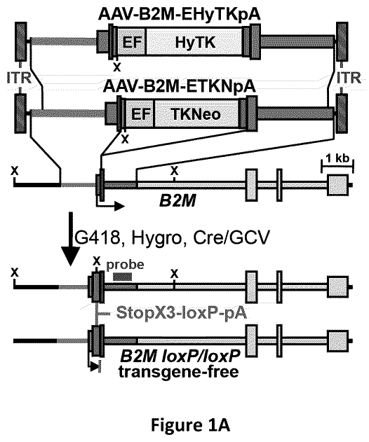

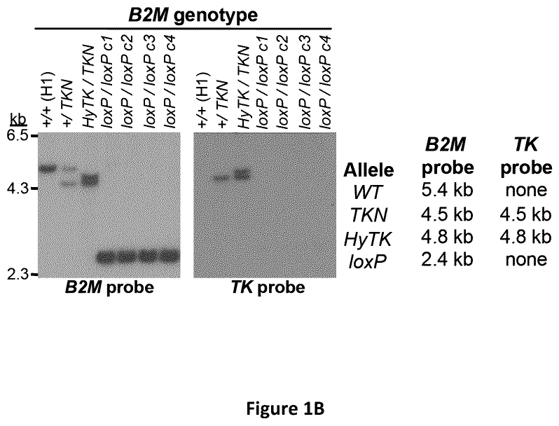

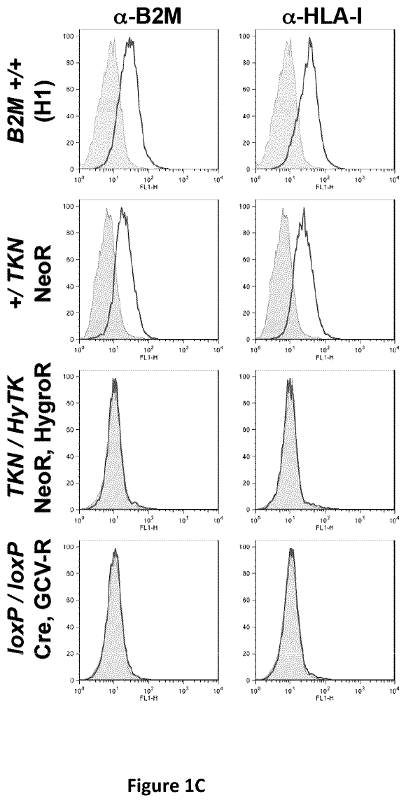

In certain embodiments, the cell genome of the B2M−/− cells may comprise no more than 100, no more than 50 or no more than 30 nucleotides of non-human DNA sequences. In certain other embodiments, the cell genome may comprise 6, 5, 4, 3, 2, 1, or 0 nucleotides of non-human DNA sequences. An exemplary technique for removing any non-human DNA introduced in disrupting the B2M gene is provided in FIG. 1A. The non-human DNA sequences can be removed by a second round of targeting to delete the HyTK or TKNeo transgenes in the first vectors or by the Cre-mediated loxP recombination.

In other embodiments, the cells instead can be engineered to recombinantly express a single chain fusion HLA class I protein in a B2M−/− genetic background. Thus, the B2M−/− cells as used herein also encompass primate, preferably human, cells that express one or more single chain fusion HLA class I proteins in a B2M−/− genetic background. The B2M−/− cells recombinantly expressing a single chain fusion HLA class I protein are nevertheless deficient in normal B2M function in that the cells do not express wild type B2M protein that form a non-covalently associated heterodimer with any HLA class I α chain on the cell surface.

The term “single chain fusion HLA class I protein,” “single chain fusion HLA class I molecule” or “single chain fusion HLA class I antigen” refers to a fusion protein comprising at least a portion of the B2M protein covalently linked, either directly or via a linker sequence, to at least a portion of an HLA-I α chain. On the other hand, the term “HLA class I protein,” “HLA class I molecule” or “HLA class I antigen” refers to a non-covalently associated heterodimer of B2M and an HLA α chain expressed on the surface of a wild type cell.

As used herein, the term “HLA class I α chain” or “HLA-I heavy chain” refers to the α chain of the HLA class I heterodimer. HLA class I heavy chain includes without limitation HLA class I α chains HLA-A, HLA-B, HLA-C, HLA-E, HLA-F, and HLA-G. Representative DNA and protein sequences are provided for HLA-A (GenBank No. K02883.1, SEQ ID NO:3; UniProt No. P01892, SEQ ID NO:4), HLA-B (NM_005514, SEQ ID NO:5; NP_005505; SEQ ID NO:6), HLA-C (NM_002117, SEQ ID NO:7; NP_002108, SEQ ID NO:8), HLA-E (NM_005516, SEQ ID NO:9; NP_005507, SEQ ID NO: 10), HLA-F (NM_018950, SEQ ID NO:11; NP_061823, SEQ ID NO:12), and HLA-G (NM_002127, SEQ ID NO:13; NP_002118, SEQ ID NO:14).

In addition, although the term “HLA class I protein/molecule” is known to refer to the MHC class I protein/molecule in human, the terms HLA and MHC are sometimes used interchangeably throughout this application: for example, the term HLA class I protein can also be used to refer to the primate equivalent to the HLA class I protein in a primate. One of skill in the art will be able to discern the meaning of the term based on the content.

The term B2M−/− cells as used herein also encompasses cells having genetically engineered disruptions in all copies of the B2M gene, wherein one B2M allele is genetically engineered to express, instead of the wild type B2M protein, a single chain fusion HLA class I protein (i.e., genetically targeted knockin in one B2M allele). B2M−/− cells with such genetic background express B2M only in the context of the single chain fusion HLA class I protein from a B2M genetic locus. In certain advantageous embodiments, the expression of the single chain fusion HLA class I protein is regulated by the endogenous B2M regulatory sequence located at the B2M locus.

In related embodiments, B2M−/− cells further encompass cells having genetically engineered disruptions in all copies of the B2M gene, wherein all B2M alleles are genetically engineered to express, instead of the wild type B2M protein, single chain fusion HLA class I proteins (i.e., genetically targeted knockin in all B2M alleles). B2M−/− cells with such genetic disruptions express B2M only in the context of single chain fusion HLA class I proteins from the genetic loci of all the alleles of the B2M gene. In certain embodiments, the cells are genetically engineered to express the same type of single chain fusion HLA class I protein from the genetic loci of all alleles of the B2M gene; while in other embodiments, the cells are genetically engineered to express different types of single chain fusion HLA class I proteins from different genetic loci of different alleles of the B2M gene.

Throughout the application, the “cells of the invention,” “isolated cells of the invention,” “B2M−/− cells,” “B2M−/− cells of the invention” or “stem cells or differentiated cells of the invention” sometimes can be used interchangeably to encompass all the B2M−/− cells described herein. In certain particular embodiments, the B2M−/− cells of the invention express a single chain fusion HLA class I protein as defined herein, in a B2M−/− background. The B2M−/− cells can be genetically engineered to express a single chain HLA class I protein either from the B2M locus or from other location of the genome. In certain particular embodiments, the cells of the invention comprise genetically engineered disruptions in all alleles of the B2M gene that preclude the expression of wild type B2M protein, and nevertheless express a single chain fusion HLA class I protein from a B2M genetic locus. In certain other particular embodiments, the cells of the invention comprise genetically engineered disruptions in all alleles of the B2M gene that preclude the expression of wild type B2M protein, and nevertheless express single chain fusion HLA class I proteins from all B2M genetic loci. The term “gene,” “allele,” and “genetic locus” may be used interchangeably throughout the application.

The “isolated cell” can be any suitable cell type for a given purpose. For example, the cell can be a pluripotent stem cell or a differentiated cell. “A stem cell” broadly encompasses any cells that are capable of further differentiation. “A pluripotent stem cell” refers to a stem cell that has the potential to differentiate into any of the three germ layers: endoderm, mesoderm or ectoderm. “An adult stem cell,” on the other hand, is multipotent in that it can produce only a limited number of cell types. “An embryonic stem (ES) cell” refers to a pluripotent stem cell derived from the inner cell mass of the blastocyst, an early-stage embryo. “Induced pluripotent stem cells (iPS cells)” are pluripotent stem cell artificially derived from a non-pluripotent cell, typically an adult somatic cell, by artificially inducing expression of certain genes.

In certain embodiments, the single chain fusion HLA class I protein comprises at least a portion of B2M and at least a portion of HLA-A, HLA-B, HLA-C, HLA-E, HLA-F or HLA-G (also referred to as a dimeric construct). In certain preferred embodiments, the HLA α chain contained in the single chain fusion HLA class I protein does not contain the leader sequence (or signal sequence) of the HLA class I α chain (leaderless HLA α chain). In certain other embodiments, the single chain fusion HLA class I protein comprises at least a portion of B2M and at least a portion of HLA-C, HLA-E or HLA-G. In certain further embodiments, the single chain fusion HLA class I protein comprises at least a portion of B2M and at least a portion of HLA-A, HLA-E or HLA-G. In certain preferred embodiments, the single chain fusion HLA class I protein comprises a leader sequence (or signal peptide) covalently linked to the at least a portion of B2M and at least a portion of an HLA α chain to ensure proper folding of the single chain fusion on the cell surface. The leader sequence can be the leader sequence of the B2M protein, the leader sequence of an HLA α chain protein or the leader sequence of other secretary proteins. In certain particular embodiments, the single chain fusion HLA class I protein comprises a B2M protein with its leader sequence removed. In certain other particular embodiments, the single chain fusion HLA class I protein comprises an HLA α chain protein with its leader sequence removed. Certain HLA class I α chains are highly polymorphic. As will be understood by those of skill in the art, the human cells and methods of the invention are applicable to any such HLA α chains and polymorphism thereof.

Single chain fusion HLA class I proteins comprising sequence variants and fragments of B2M and/or HLA α chains are contemplated by the instant invention, wherein such single chain fusion constructs nevertheless possess normal HLA class I functions, e.g., forming proper secondary structure of the heterodimer on the cell surface, presenting peptides in the peptide binding cleft and engaging the inhibitory receptors on the surface of NK cells. In certain embodiments, the variants share at least 75%, 80%, 81%, 85%, 88%, 90%, 91%, 92%, 93%, 94%, 95%, 96%, 97%, 98%, 99%, or complete sequence homology with the naturally occurring HLA heavy chains and B2M sequences, wherein the variants possess normal HLA class I functions. In certain other embodiments, the variants share at least 75%, 80%, 81%, 85%, 88%, 90%, 91%, 92%, 93%, 94%, 95%, 96%, 97%, 98%, 99%, or complete sequence homology with the sequences of B2M or HLA heavy chains as shown in SEQ ID NOs:2, 4, 6, 8, 10, 12 or 14.

In certain particular embodiments, the HLA-A variants share at least 85%, 88%, 90%, 91%, 92%, 93%, 94%, 95%, 96%, 97%, 98%, 99%, or complete sequence homology with SEQ ID NO:4. In certain other particular embodiments, the HLA-B variants share at least 81%, 83%, 85%, 88%, 90%, 91%, 92%, 93%, 94%, 95%, 96%, 97%, 98%, 99%, or complete sequence homology with SEQ ID NO:6. In certain further embodiments, the HLA-C variants share at least 90%, 91%, 92%, 93%, 94%, 95%, 96%, 97%, 98%, 99%, or complete sequence homology with SEQ ID NO:8. In yet other embodiments, the HLA-E variants share at least 97%, 98%, 99%, or complete sequence homology with SEQ ID NO:10. In certain particular embodiments, the HLA-F variants share at least 99%, or complete sequence homology with SEQ ID NO:12. In certain other embodiments, the HLA-G variants share at least 98%, 99%, or complete sequence homology with SEQ ID NO: 14.

In certain other embodiments, the single chain fusion HLA class I protein comprises a full length B2M (including its leader sequence) and an HLA α chain without the leader sequence (leaderless HLA α chain); while in certain other embodiments, the single chain fusion HLA class I protein comprises a B2M protein without the leader sequence. It is understood that B2M−/− cells expressing two, three or more different types of single chain fusion HLA class I protein in any combination, for example, expressing SC fusion comprising HLA-A (or a leaderless HLA-A) and SC fusion comprising HLA-C (or a leaderless HLA-C), expressing SC fusion comprising HLA-A (or a leaderless HLA-A) and SC fusion comprising HLA-E (or a leaderless HLA-E), or expressing SC fusion comprising HLA-B (or a leaderless HLA-B), SC fusion comprising HLA-E (or a leaderless HLA-E) and SC fusion comprising HLA-G (or a leaderless HLA-G), etc., are all contemplated by the invention.

Natural killer (NK) cells are part of the innate immune response. Several pathogens can down regulate HLA class I protein expression in infected cells. The NK cells monitor infection by recognizing and inducing apoptosis in cells that do not express HLA class I proteins. The inhibitory receptors on the NK cell surface recognize HLA class I α chain alleles thereby preventing NK-medicated apoptosis in uninfected normal cells. Thus, in certain particular embodiments, the single chain fusion HLA-I protein inhibits NK cell-mediated killing of cells that do not express endogenous HLA class I proteins by binding to the inhibitory receptors on the NK cells. For example, HLA-E is a ligand for the CD94/NKG2 receptor of NK cells that inhibits NK cell-mediated apoptosis. Thus, in certain particular embodiments, the B2M−/− cell expresses the single chain fusion HLA class I protein comprising at least a portion of B2M and at least a portion of HLA-E. In addition, HLA-G is normally expressed on the surface of placental cytotrophoblasts that do not express HLA-A, B or C, and it protects these cells from NK cell-mediated lysis by interacting with the inhibitory ILT2(LIR1) receptor on NK cells (Pazmany et al., 1996, Science 274, 792-795). Thus, in certain other preferred embodiments, the B2M−/− cell expresses the single chain fusion HLA class I protein comprising at least a portion of B2M and at least a portion of HLA-G.

In certain particular embodiments, the single chain fusion HLA class I protein comprises at least a portion of B2M and at least a portion of HLA-A0201, an allele of HLA-A. HLA-A0201 (SEQ ID NO:4) is a common HLA class I allele found in a large percentage of the population in the United States. Thus, in certain advantageous embodiments, the isolated cell expresses the single chain fusion HLA class I protein comprising at least a portion of B2M and at least a portion of HLA-A0201 in a B2M−/− genetic background, wherein the isolated cell is immune compatible with a large percentage of the human population in the United States. Other suitable common alleles that can be used include without limitation HLA-A0101, HLA-A0301, HLA-B0702, HLA-B0801, HLA-C0401, HLA-C0701, and HLA-C0702. In certain preferred embodiments, the HLA allele comprises at least a portion of HLA-A0201 (SEQ ID NO:4), HLA-B0702 (SEQ ID NO:6) or HLA-C0401 (SEQ ID NO:8).

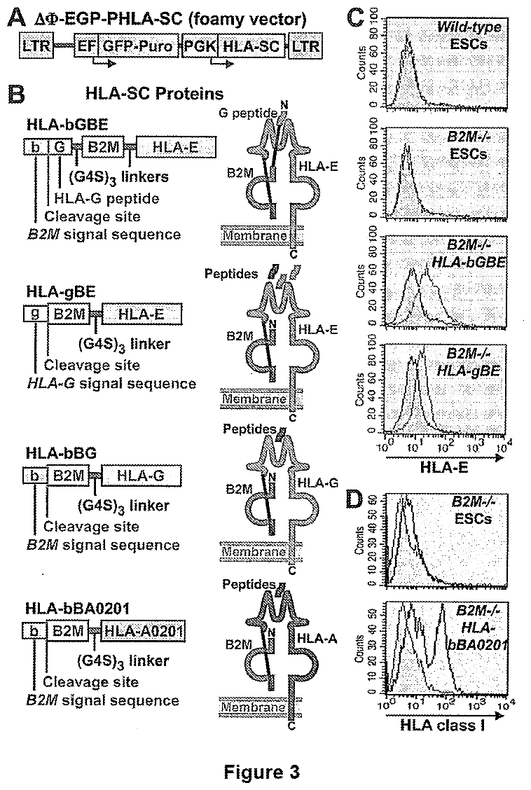

In certain further embodiments, the single chain fusion HLA class I protein also comprises a specific peptide antigen that occupies the peptide binding cleft of the single chain fusion HLA class I protein, wherein the peptide antigen is covalently linked to the single chain fusion HLA class I protein (also referred to as a trimeric construct). An example of the trimeric construct is shown in FIG. 3B. The HLA-bGBE construct of FIG. 3B comprises B2M and HLA-E covalently linked to a peptide antigen (such as, but not limited to, the HLA-G peptide antigen as illustrated in the figure) (SEQ ID NO:23) designed to occupy the peptide binding cleft of the single chain fusion HLA class I protein. In certain other embodiments, the covalently linked peptide antigen is cleaved via a built in protease cleavage site, and the cleaved peptide antigen can bind to the peptide binding cleft of the single chain fusion HLA-I protein for presentation. In certain alternative embodiments, the peptide antigen occupying the peptide binding cleft of the single chain fusion HLA class I protein is produced by the intracellular antigen processing pathway, in which the peptide antigen is produced by proteasome, transported to and loaded onto the single chain fusion HLA class I protein in the endoplasmic reticulum. In certain particular embodiments, the peptide antigen comprises a peptide of a tumor antigen. In certain other embodiments, the peptide antigen comprises a peptide of a protein from a pathogen including without limitation a bacterium, a virus, a fungus and a parasite. In further embodiments, the peptide antigen comprises a peptide of a tumor antigen. In certain particular embodiments, the B2M−/− cell expresses a single chain fusion HLA class I protein that is covalently linked to a peptide that does not comprise an auto-antigen or neo-antigen to the patient. It is within the ability of a skilled person to design the single chain fusion HLA class I protein and the peptide antigen presented thereon to modulate the immune response that may be elicited in a recipient.

The isolated B2M−/− cell expressing a single chain fusion HLA class I protein comprising a specific peptide antigen either covalently or non-covalently bound to the single chain fusion HLA class I protein can be used, for example, for administration to a recipient to elicit an immune response. Accordingly, in a related aspect, the invention provides a vaccine comprising the isolated cell of the invention, wherein the vaccine is capable of eliciting in a recipient an immune response specific for the target peptide antigen. The immune response includes without limitation a cellular immune response and/or a humoral immune response. The vaccine may comprise a stem cell or a differentiated cell; in certain particular embodiments, the cell is a differentiated dendritic cell. In certain other embodiments, the cell further expresses a cytokine. Any suitable cytokine can be used; in certain particular embodiments, the cytokine is IL2 or IFN-γ. In certain preferred embodiments, the cell is a human cell and the recipient is a human.

The single chain fusion HLA class I protein can be expressed from an expression vector that allows either transient or more preferably, stable expression of the protein in a B2M−/− cell. Exemplary suitable expression vectors are known in the art. One such example is a retroviral vector, which is capable of integrating into the cellular genome to provide long-term, stable expression of an exogenous gene. In certain particular embodiments, the viral vector is derived from human foamy virus, a type of retrovirus. Other suitable viral vectors include without limitation vectors derived from retrovirus, adenoviral virus, adeno-associated virus, lentivirus, herpes simplex virus, vaccinia virus, and pox virus.

In certain preferred embodiments, the polynucleotide capable of encoding a single chain fusion HLA class I protein is integrated into the chromosome of the cells, preferably into the B2M or the HLA loci, for stable expression. Thus, in certain preferred embodiments, the B2M loci are disrupted by inserting in the B2M loci the polynucleotide capable of encoding a single chain fusion HLA class I protein to replace the expression of the endogenous wild type B2M protein. The result of such gene targeting disrupts normal B2M expression and precludes formation of wild type HLA class I proteins but permits expression of a predetermined single chain fusion HLA class I protein of choice on the surface of the otherwise B2M deficient cells. Other expression vectors are also contemplated and the selection of suitable expression vector is within the ability of one ordinary skill in the art.

According to the vector design, the polynucleotide capable of expressing a single chain fusion HLA class I protein is delivered to a cell by viral infection (when a viral vector is used) or by other delivery methods including without limitation transfection, electroporation, gene targeting or liposome-mediated DNA delivery.

Any immune effects of the single chain fusion HLA class I protein expressing B2M−/− cells can be studied by various means. For example, B2M−/− cells expressing a SC fusion HLA class I protein can be differentiated into antigen-presenting dendritic cells (iDCs). Suppression of NK cell-mediated lysis can be measured by chromium release assays after incubating iDCs with normal human NK cells and NKL cell lines. A variety of controls (untransduced B2M−/− iDCs, B2M+/+ iDCs, the 721.221 class I-negative cell line, and anti-receptor and anti-HLA antibodies) can be used to establish the specificity of the interactions. Additional characterization can be done with Elispot assays by incubating the cells with T cells.

In a related aspect, the invention provides an HLA class I-typed B2M−/− cell bank, wherein the cells of the cell bank comprise a B2M−/− genetic background and are engineered to express one or more types of single chain fusion HLA class I proteins in which the HLA α chain is selected from the group consisting of HLA-A, HLA-B, HLA-C, HLA-E, HLA-F and HLA-G. In certain embodiments, the cell bank comprises a population of cells that expresses a single chain fusion HLA class I protein in which the HLA α chain comprises HLA-A. In certain other embodiments, the cell bank comprises a population of cells that expresses a single chain fusion HLA class I protein in which the HLA α chain comprises HLA-B. In certain further embodiments, the cell bank comprises a population of cells that expresses a single chain fusion HLA class I protein in which the HLA α chain comprises HLA-C. In yet other embodiments, the cell bank comprises a population of cells that expresses a single chain fusion HLA class I protein in which the HLA α chain comprises HLA-E. In certain other embodiments, the cell bank comprises a population of cells that expresses a single chain fusion HLA class I protein in which the HLA α chain comprises HLA-F. In certain particular embodiments, the cell bank comprises a population of cells that expresses a single chain fusion HLA class I protein in which the HLA α chain comprises HLA-G. In certain particular embodiments, the cell bank comprises the above-described one or more or preferably all populations of cells.

The cells of the cell bank can be pluripotent stem cells or differentiated cells. In certain particular embodiments, the cell bank comprises different types of differentiated cells, such as skin cells, pancreatic beta islet cells, etc., that express the same single chain fusion HLA class I protein. While in other particular embodiments, the cell bank comprises different types of differentiated cells, such as skin cells, pancreatic beta islet cells, etc., that each express different single chain fusion HLA class I proteins. It can be determined by a skilled researcher or clinician to choose suitable donor cells from the cell bank for a given patient. In certain other embodiments, some of the cells of the cell bank express the HLA class I allele that matches the HLA class I allele of the patient to whom the cells are administered. In certain preferred embodiments, the cell is a human cell and the patient is a human. In certain particular embodiments, the cells express a single chain fusion HLA class I protein comprising B2M and HLA-A0201 that matches the HLA allele of a large portion of the population in the United States.

In another aspect, the invention provides a method of transplantation in a patient in need thereof comprising the step of administering to the patient an effective amount of the cells of the invention for transplantation. Because the B2M−/− cells do not express wild type HLA class I protein on the cell surface, the cells when administered to a patient elicit minimal or no immune responses in the patient. Thus, transplantation using the B2M−/− cells limits the need for taking immune suppressant therapies. Thus, in certain preferred embodiments, the patient is immune competent. In certain other embodiments, the cell is an isogeneic cell; while in other embodiments, the cell is an allogeneic cell.

In certain further embodiments, the cells of the invention are pluripotent stem cells; while in other embodiments, the cells of the invention are differentiated cells. In certain preferred embodiments, the cell is a human cell and the patient is a human patient. In certain particular embodiments, the method of transplantation comprises administering to a human an effective amount of the pluripotent stem cells or differentiated cells. In certain preferred embodiments, the cells of the invention further express one or more engineered single chain fusion HLA class I proteins. In certain other embodiments, the cells are able to escape NK cell-mediated killing and elicit minimal or no immune response in the recipient after transplantation.

Transplantation therapy, replacement therapy or regenerative therapy refers to therapies for a disease condition by administering to a patient cells or tissues to replenish or replace defective cellular functions in a target organ. In certain particular embodiments, the need for transplantation arises as a result of physical or pathological injuries to a tissue or organ. In certain other particular embodiments, the need for transplantation arises as a result of one or more genetic defect or mutation in the patient and the transplantation of the cells of the invention replenishes or replaces defective cellular functions in the patient without the need for gene therapy to correct the underlying genetic mutation of the patient. In certain further embodiments, the transplantation includes without limitation hematopoietic stem cell transplantation, or transplantation of cells that are incorporated into an organ such as liver, kidney, pancreas, lung, brain, muscle, heart, gastrointestinal tract, nervous system, skin, bones, bone marrow, fat, connective tissue, immune system, or blood vessels. In certain particular embodiments, the target organ is a solid organ.

In certain particular embodiments, the cells administered to the recipient may or may not be incorporated into an organ in need of such therapy. In certain embodiments, the cells of the invention are differentiated into the desired cell type, either before or after transplantation, and provide the necessary cellular function without itself being incorporated into the tissue at the site of transplantation. For example, in certain embodiments for treating diabetes, the cells of the invention either as pluripotent stem cells or differentiated pancreatic beta islet cells are transplanted to a diabetic patient. The transplanted cells need not reconstitute a functioning pancreas: they just need to secrete insulin in response to glucose levels. In certain particular embodiments, the cells are transplanted into an ectopic location and are not fully incorporated into the pancreas. Transplantation of pluripotent cells of the invention, differentiated cells of the invention, or a tissue differentiated and developed ex vivo from the cells of the invention are all contemplated by the invention. In certain preferred embodiments, the cell is a human cell and the patient is a human patient. In certain other preferred embodiments, the cells of the invention express one or more single chain fusion HLA class I proteins.

In a further aspect, the invention provides a method of treating a disease condition in a patient in need thereof comprising the step of administering to the patient an effective amount of the cell of the invention to treat the disease condition, wherein the disease condition is diabetes, an autoimmune disease, cancer, infection, anemia, cytopenia, myocardial infarction, heart failure, skeletal or joint condition, osteogenesis imperfecta or burns. In certain particular embodiments, the disease condition results from pathological or physical injuries to a tissue or organ. In certain embodiments, the cells of the invention are stem cells; while in other embodiments, the cells of the invention are differentiated cells. In certain preferred embodiments, the cell is a human cell and the patient is a human patient. In certain particular embodiments, the human cell is a differentiated cell. Transplantation of a tissue developed ex vivo from the cells of the invention is also contemplated by the invention. In certain preferred embodiments, the cells of the invention further express one or more single chain fusion HLA class I proteins. In certain embodiments, the cell is an isogeneic cell; while in other embodiments, the cell is an allogeneic cell.

In certain particular embodiments, the cell is a differentiated cell including without limitation a dendritic cell, lymphocyte, red blood cell, platelet, hematopoietic cell, pancreatic islet cell, liver cell, muscle cell, keratinocyte, cardiomyocyte, neuronal cell, skeletal muscle cell, ocular cell, mesenchymal cell, fibroblast, lung cell, GI tract cell, vascular cell, endocrine cell and adipocyte. In certain other particular embodiments, the invention provides a method of treating a disease condition in a solid organ. In certain embodiments, the cells of the invention used in treating a disease condition express one or more single chain fusion HLA class I proteins.

“Treating” a patient having a disease or disorder means accomplishing one or more of the following: (a) reducing the severity of the disease; (b) arresting the development of the disease or disorder; (c) inhibiting worsening of the disease or disorder; (d) limiting or preventing recurrence of the disease or disorder in patients that have previously had the disease or disorder; (e) causing regression of the disease or disorder; (f) improving or eliminating the symptoms of the disease or disorder; and (f) improving survival. In certain preferred embodiments, the disease or disorder is a disease or disorder that can be treated by transplantation of tissues or cells.

The effective amount of the isolated cells of the invention for transplantation or for treating a disease condition depends on a number of factors, such as the type of tissue, the severity of the disease condition, the transplantation reaction, the reason for transplantation, and the age and general health of the patient. The effective amount can be determined by a skilled researcher or clinician by routine practice. Due to the reduced immunogenicity of the transplanted cells, relative large amount of cells can be tolerated by a patient to achieve the desired therapeutic effects. Alternatively, the cells can be repeatedly transplanted at intervals until a desired therapeutic effect is achieved.

The route for administration of the cells of the invention is not limited in any particular way. Exemplary delivery routes include without limitation intravenous, intramuscular, subdermal, intraperitoneal, transcutaneous, intracutaneous, and subcutaneous route. The cells of the present invention can also be administered topically by injection. For example, the cells can be injected into an injured joint, a fractured bone, an infarct site, an ischemic site or their periphery.

In certain particular embodiments, the cells are administered via a delivery device including without limitation a syringe. For example, the cells can be suspended in a solution or a pharmaceutical composition contained in such a delivery device. The “solution” or “pharmaceutical composition” comprises a physiological compatible buffer and optionally a pharmaceutically acceptable carrier or diluent in which the cells of the invention remain viable. The use of such carriers and diluents is well known in the art. The solution includes without limitation physiologically compatible buffers such as Hank's solution, Ringer' solution, or physiologically buffered saline. The cells can be kept in the solution or pharmaceutical composition for short term storage without losing viability. In certain particular embodiments, the cells are frozen for long term storage without losing viability according to cryopreservation methods well-known in the art.

Aqueous injection suspensions may contain substances which increase the viscosity of the suspension, such as sodium carboxymethyl cellulose, sorbitol, or dextran, but still fluid to the extent that can be easily delivered by syringe injection. The solution is preferably sterile, stable under the conditions of manufacture and storage and is free of microorganism contamination through the use of, for example, parabens, chlorobutanol, phenol, ascorbic acid, thimerosal, and the like. The cells contained in the solution can be stem cells or differentiated cells as described herein, in a pharmaceutically acceptable carrier or diluent and, as required, other ingredients indicated above.

The cells may be administered systemically (e.g., intravenously) or locally (e.g., directly into a myocardial defect under the guidance of echocardiogram, or by direct application to damaged tissues or organs accessible during open surgery). For injections, the cells may be in an injectable liquid suspension preparation or in a biocompatible medium which is injectable in liquid form and becomes semi-solid at the site of damaged tissue. A syringe, a controllable endoscopic delivery device or other similar devises can be used so long as the needle lumen is of sufficient diameter (e.g. at least 30 gauge or larger) to avoid physical damages to the cells during delivery.

In certain other embodiments, the cells can be transplanted via a solid support, e.g., a planar surface or three-dimensional matrix. The matrix or planar surface is surgically implanted into the appropriate site in a patient. For example, a patient needing a pancreatic graft can have differentiated cells on a solid support surgically implanted in the pancreas tissue. Exemplary solid support includes without limitation a patch, a gel matrix (such as GELFOAM® from Pharmacia-Upjohn), polyvinyl alcohol sponge (PVA)-collagen gel implants (such as IVALON, Unipoint Industries, High Point, NC) and other similar or equivalent devices. A variety of other encapsulation technologies can be used with the cells of the invention, for example, WO 91/10470; WO 91/10425; U.S. Pat. No. 5,837,234; U.S. Pat. No. 5,011,472; U.S. Pat. No. 4,892,538).

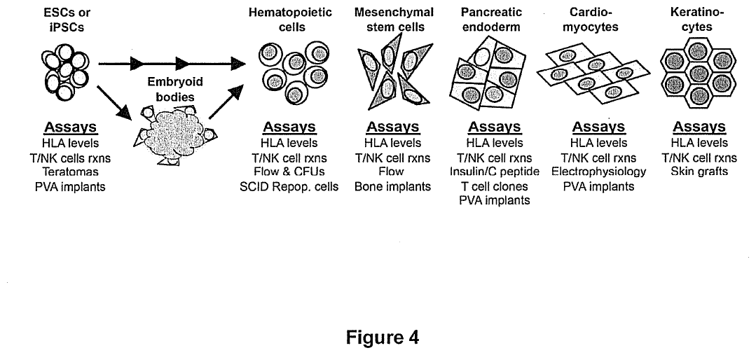

The cells of the invention can be differentiated into various cell types of all three lineages, including without limitation hematopoietic, mesenchymal, pancreatic endoderm, cardiac and keratinocytes cells. In certain embodiments, the differentiated cell further expresses a single chain fusion HLA class I protein. In general, each cell type can be analyzed for HLA class I protein expression, reactivity with human T cells and NK cells, appropriate differentiation markers, and xenotransplantation in immunodeficient mice to examine in vivo developmental potential. See FIG. 4. A brief discussion of each differentiated cell type follows.

In certain embodiments, the cells of the invention can be differentiated to hematopoietic cells for treating various hematopoietic diseases currently treated by bone marrow transplantation. Patients receiving transfusion can become refractory to platelet transfusions due to HLA mismatches. Anemic or cytopenic patients can be treated by delivering the cells of the invention-derived erythrocytes, platelets or neutrophils to treat bleeding or infection.

Further, stem cells of the invention-derived dendritic cells are antigen-presenting cells that can be used as cellular vaccines when properly engineered. In certain embodiments, the cells of the invention engineered to express a single chain fusion HLA class I protein and a unique peptide antigen are used to vaccinate against specific pathogen or tumor antigens. In certain other embodiments, differentiated B2M−/− cytotoxic lymphocytes with HLA-restricted reactivity against specific antigens are used to eliminate infected cells or tumor cells.

To obtain hematopoietic cells, the pluripotent cells are first allowed to form embryoid bodies, thereafter non-adherent cells were cultured in the presence of hematopoietic cytokines to develop into specific cell lineages. The differentiation of hematopoietic cells from the cells of the invention that express a single chain fusion HLA class I protein expressers can be analyzed by flow cytometry and colony assays. The different cell populations are sorted based on their surface markers, and used to monitor the expression of HLA genes and reactivity with human NK cells and T cells as measured by Elispot, mixed lymphocyte reactions, and cytotoxicity assays. The effectiveness of the single chain fusion HLA constructs on suppression of NK cell-mediated killing can be examined at different stages of differentiation and transplantation. See Bix et al., 1991, Nature 349, 329-331. The hematopoietic stem cells can also be assayed using xenotransplantation models in, for example, immunodeficient mice (SCID-repopulating cells or SRCs).

The cells of the invention can be differentiated into hematopoietic cell either before or after the cells are administered to a patient. In certain preferred embodiments, the cell is a human cell and the patient is a human. In vitro hematopoietic differentiation can be performed according to established protocols. See for example, Slukvin et al., 2006, J Immunol 176:2924-32, and Chang et al., 2006, Blood 108:1515-23.

In certain other embodiments, the cells of the invention can be differentiated into mesenchymal stem cells. In certain embodiments, the cells of the invention express one or more single chain fusion HLA class I proteins. MSCs have the potential to form several differentiated cell types, including marrow stromal cells, adipocytes, osteoblasts, and chondrocytes. Thus, inducing pluripotent stem cells to form MSCs (iMSCs) is useful in treating skeletal and joint conditions. The iMSCs can be further differentiated into osteoblasts and formed bone in vivo. Deyle et al., 2012, Mol Ther. 20(1):204-13. Cellular responses of T cells and NK cells to ESCs, iMSCs, and their more terminally differentiated derivatives such as osteoblasts can be examined.

In certain particular embodiments, the mesenchymal stem cells are capable of differentiating into non-limiting examples of cell types such as marrow stromal cells, adipocytes, osteoblasts, osteocytes and chondrocytes. The cells of the invention are differentiated into mesenchymal stem cells either before or after the cells are administered to a patient. In certain preferred embodiments, the cell is a human cell and the patient is a human. In vitro mesenchymal differentiation can be performed according to established protocols. See for example, Deyle et al., supra.

In yet other particular embodiments, the cells of the invention can be differentiated into insulin-producing pancreatic islet cells. In certain embodiments, the cells of the invention express one or more single chain fusion HLA class I proteins. The cells of the invention can be used to treat insulin-dependent diabetes mellitus. Advantageously, the transplanted cells do not need to reconstitute a functioning pancreas: they just need to secrete insulin in response to glucose levels. Therefore the treatment can succeed with different cell doses, with cells that are not perfectly differentiated into adult cell types, and when cells are transplanted into an ectopic location. Specific auto-antigens such as those derived from GAD65 or Insulin can cause autoimmune destruction of β cells in diabetes (Di Lorenzo et al., 2007, Clin Exp Immunol 148, 1-16). Thus, B2M−/− cells or B2M−/− cells expressing a single chain fusion HLA class I protein presenting a predetermined peptide antigen provide additional advantages in that they do not present these auto-antigens and can avoid autoimmune rejection and prevent a relapse of diabetes after transplantation.

The cells of the invention can be differentiated into pancreatic cells as described previously, which employs exposure of cells to different cytokines and drugs to promote sequential formation of mesendoderm, definitive endoderm, and pancreatic progenitors (Kroon et al., 2008, Nat Biotechnol 26, 443-452). These cells can be further cultured in implants in immunodeficient mice. The cells of the invention with or without expressing a single chain fusion HLA class I protein and wild-type cell lines can be analyzed at different developmental stages for their reactivity with T cells and NK cells.

The cells of the invention are differentiated into pancreatic islet cell either before or after patient administration. In certain preferred embodiments, the cell is a human cell and the patient is a human. In vitro hematopoietic differentiation can be performed according to established protocols. See for example, Kroon et al., 2008, Nat Biotechnol 26, 443-452.

In certain other particular embodiments, the cells of the invention can be differentiated into cardiomyocytes. In certain embodiments, the cells of the invention further express one or more single chain fusion HLA class I proteins. The common clinical problems of myocardial infarction and congestive heart failure can be treated by transplanting healthy stem cell-derived cardiomyocytes that engraft and re-establish functional myocardium. The cells of the invention-derived cardiomyocytes allow these treatments to proceed with pre-packaged cells and avoid the immunosuppression currently required for allogeneic heart transplants. Physiologically relevant tests can be performed on the cardiomyocytes derived from the cells of the invention, such as electrical conduction and contraction studies. B2M−/− stem cells or differentiated cardiomyocytes with or without expressing a single chain fusion HLA class I protein can be tested to determine their immunological reactivity when expressing cardiomyocyte genes, and to establish which HLA modifications minimize these immune responses.

The cells of the invention can be differentiated into cardiomyocytes either before or after the cells are administered to a patient. In certain preferred embodiments, the cell is a human cell and the patient is a human. In certain embodiments, the cells of the invention are differentiated into cardiomyocytes for treating diseases including without limitation myocardial infarction and congestive heart failure. In vitro cardiomyocyte differentiation can be performed according to established protocols. See for example, Laflamme et al., 2007, Nat Biotechnol 25, 1015-1024.

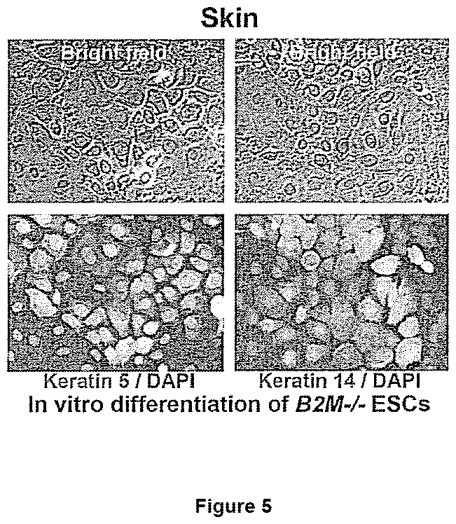

In yet other particular embodiments, the cells of the invention can be differentiated into keratinocytes. In certain embodiments, the cells of the invention used for differentiation into keratinocytes express one or more single chain fusion HLA class I proteins. Severe burns and genetic skin conditions require treatment with skin grafts, and this is currently done with a variety of cell sources such as porcine skin grafts and cultured autologous human keratinocytes. Keratinocytes derived from the cells of the invention can provide a major clinical advance, since burns could be treated as an emergency with pre-packaged cells, and genetic diseases such as epidermolysis bullosum can be treated with normal cells (albeit with the B2M−/− background in the cellular chromosome) that do not require correction of the responsible genetic mutations. In many cases the cells only need to engraft long enough for neighboring host cells to repopulate the affected area. FIG. 5 shows in vitro differentiation of Keratin 5+ and Keratin 14+ keratinocyte colonies from the cells of the invention. The cells of the invention were cultured in matrigel cultures, followed by expansion in serum-free keratinocyte medium containing all-trans retinoic acid and BMP4 as described previously (Itoh et al., 2011, PNAS USA 108, 8797-8802). For in vivo differentiation, the cells of the invention can be embedded in polyvinyl alcohol sponge (PVA)-collagen gel implants for transplantation into a recipient. The cells of the invention can be differentiated into keratinocytes either before or after transplantation. In certain preferred embodiments, the cell is a human cell and the patient is a human.

In yet another aspect, the invention provides a use of the cells of the invention for the preparation for a medicament for transplantation. In a related aspect, the invention provides a use of the cells of the invention for the preparation for a medicament for treating a disease condition.

Further, the cells of the invention can serve as a research tool to provide a system for studying the functions of immunoregulatory proteins in a B2M−/− genetic background. In certain embodiments, the cells of the invention further express one or more single chain fusion HLA class I proteins. Accordingly, in a related aspect, the invention provides a method of determining the function of an immunoregulatory protein comprising the steps of introducing one or more immunoregulatory genes into the cells of the invention of the invention and assaying for the activities of the immunoregulatory genes. In certain preferred embodiments, the cell is a human cell. For example, the cells of the invention can be used to study the function of an immune regulatory gene, or to study an immune response, in the absence of unwanted class I antigens. In certain embodiments, the cells of the invention express HLA-F or a single chain fusion HLA class I protein comprising B2M and HLA-F, wherein the function of the HLA-F can be studied in the B2M−/− background. In a further related aspect, the invention provides a method of identifying a compound or molecule that modulates the function of the immunoregulatory protein comprising the steps of contacted the B2M−/− cells comprising the one or more immunoregulatory genes with a compound or molecule of interest and assaying for the activities of the immunoregulatory genes. In certain preferred embodiments, the cell is a human cell.

In yet another related aspect, the invention provides an in vivo research tool in a mammal, particular in a non-human primate, that are administered the cells of the invention, for studying the functions of immunoregulatory genes, or identifying a compound that modulates the function of an immunoregulatory gene in the administered cells in a B2M−/− genetic background. In certain embodiments, the cells of the invention further express one or more single chain fusion HLA class I proteins.

Mice, especially immune deficient mice, have been used as a model system for studying human cells in vivo. Human stem cells can behave differently in mice. In addition, the mouse and human immune systems have different NK cell receptors and non-classical MHC class I genes (e.g. HLA-E, F and G). Therefore, a Macaca nemestrina (Mn, pigtailed macaque) model can be developed to study the cells of the invention. The Macaca mulatta genome has been sequenced, which is highly homologous to the nemestrina genome. Further, the organization of macaque MHC loci is similar to human HLA, including the non-classical genes. Homologs of the human HLA-E and HLA-G genes have been identified in macaques. The macaque MHC loci also contain homologs of many human NK cell receptors. Human B2M−/− ESCs as well as Mn B2M−/− ESCs can be used for transplantation in macaques.

MHC class I-deficient (B2M−/−) macaque ESCs can be developed using the same AAV-mediated gene targeting strategy described for human cells. Mn versions of the single-chain HLA class I fusion proteins are expressed in the B2M−/− macaques ESCs using the analogous viral vectors as described above.

Cells can be expanded in vitro and labeled with a vector expressing GFP for subsequent identification of transplanted cells. The cells can be embedded in polyvinyl alcohol sponge (PVA)-collagen gel implants, and placed subdermally into macaques. The implants can be harvested, sectioned and stained to determine the cell types that are present. Specific antibodies can be used to identify the differentiated cell types formed by the transplanted cells.

Any and every embodiment described above applies to any and every aspect of the invention, unless the context clearly indicates otherwise. All embodiments within and between different aspects can be combined unless the context clearly dictates otherwise.

The Examples, which follow, are illustrative of specific embodiments of the invention, and various uses thereof. They are set forth for explanatory purposes only, and are not to be taken as limiting the invention.

### EXAMPLES

### Example 1 Construction of Human Pluripotent Stem Cells with Knockout Mutations in B2M Genes

Human pluripotent stem cells were created with knockout mutations in both alleles of the beta-2 microglobulin (B2M) genes that encodes the common subunit required for surface expression of all HLA class I heterodimers (HLA-A, B, C, E, F and G). Adeno-associated virus (AAV) gene targeting vectors were used to construct B2M−/− (class I-negative) H1 human ESCs (University of Wisconsin). Human pluripotent stem cells were infected with AAV gene targeting vectors and the B2M gene was inactivated by homologous recombination. AAV mediated gene targeting methodology has been described previously in for example, Khan et al., 2011, Protocol, 482:482-501 and Khan et al., 1990, Science 248:1227-30. These references are hereby incorporated by reference in their entirety.

FIG. 1 describes the construction of HLA class I-negative human H1 ESCs cells using the adeno-associated virus (AAV) gene targeting vectors. The two AAV vectors used contain homologous arms surrounding exon 1 of the human B2M gene, and are designed to insert either a TKNeo or HyTK fusion gene encoding G418 or hygromycin resistance respectively into exon 1 (FIG. 1A). The H1 ESCs were infected with the AAV-B2M-ETKNpA vector and 30% of G418-resistant cells were targeted at one B2M allele based on Southern blot analysis. One of these clones was then infected with the AAV-B2M-EHyTKpA vector and 10% of hygromycin-resistant cells were targeted at B2M. Southern blot analysis of a representative clone that had deletions in both B2M alleles (B2M−/−) is shown in FIG. 1B (HyTK/TKN). None of the targeted clones analyzed contained random integrants. The sequences of the targeting vector plasmid pA2-B2METKMpA and pA2-B2MEHuTKpA are shown in SEQ ID NO:21 and SEQ ID NO:22, respectively.

Cre recombinase was then used to remove the floxed TKNeo and HyTK transgenes from the B2M loci. Cre was delivered transiently by a non-integrating foamy virus vector, which is a type of retroviral vector previously described that efficiently infects human ESCs. See Deyle, et al., 2010, J. Virol 84, 9341-9 and Gharwan et al., 2007, Mol Ther 15, 1827-1833. Four clones lacking the TKNeo and HyTK transgenes were selected by gancyclovir section that kills cells expressing thymidine kinase (TK). The results shown by Southern blot analysis demonstrated transgene-free double knockouts (FIG. 1B).

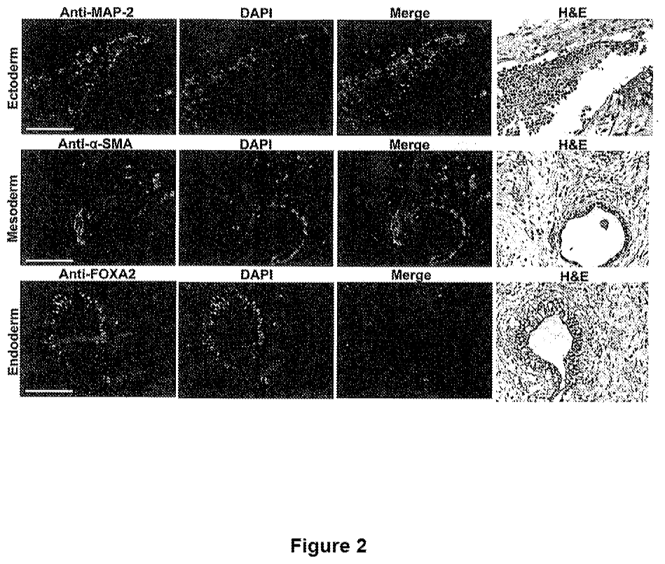

Karyotypes were checked on two of these clones and found to be normal (data not shown), and teratoma assays conducted in immunodeficient mice showed that these cells had trilineage developmental potential (FIG. 2). Flow cytometry with antibodies against B2M (anti-B2M-01-PE from SantaCruz Biotechnology) and pan-HLA class I antigens (W6/32 from Sigma-Aldrich) confirmed that these cells did not express HLA class I proteins on the cell surface (FIG. 1C).

### Example 2 Expression of Single Chain Fusion HLA Class I Proteins in B2M Knockout Cells

In mice, HLA class I-negative cells can be destructed by Natural Killer (NK) cells through the “missing self” mechanism. Bix et al., 1991, Nature 349, 329-331. Human NK cells have different receptors, but an analogous inhibition of NK cell killing is mediated through interactions of NK cell receptors with HLA-C, E and G. The “missing self” phenomenon has largely been described for class I-deficient hematopoietic cells, and the mouse transplantation data reported previously showing that many types of B2M−/− organs survived in B2M+/+ hosts suggests that it may be less important when transplanting cells form solid organs. However, given that it could significantly affect donor cell survival in some settings, specific HLA class I genes as single chain fusion proteins that suppress NK cell killing were introduced to the B2M−/− cells.

The strategy for expressing specific HLA class I genes in a B2M−/− background is shown in FIG. 3. The B2M chain was fused to the specific HLA class I heavy chain, thereby allowing surface expression of the class I chain even in B2M−/− cells. Integrating foamy virus vectors were used to express these single chain fusion proteins. Foamy virus vectors are a type of retroviral vector with a large packaging capacity that can efficiently infect human pluripotent stem cells (Gharwan at al., supra). One such representative single chain fusion HLA class I protein foamy virus construct is shown in FIG. 3A. The ΔΦ-EGP-PHLA-SC foamy vectors included a GFP-Puro fusion protein gene driven by an EF1 alpha promoter to allow for puromycin selection and GFP expression in transduced cells, and a separate expression cassette with a ubiquitously expressed PGK promoter driving an HLA single chain fusion (HLA-SC) construct (FIG. 3A). The vector design produced constitutive expression of both transgenes, but many other vector designs and internal promoters can also be used. For example, the GFP-Pur gene can be driven by the pGK promoter, and the EFlalpha promoter controls the expression of the SC HLA gene.

As shown in FIG. 3B, the HLA-bGBE trimeric single chain fusion construct included a covalently attached HLA-G peptide (SEQ ID NO:23) in the HLA-E peptide binding cleft, while the HLA-gBE dimeric construct included an HLA-G signal peptide that was cleaved off but still bound non-covalently to the HLA-E molecule. See Crew et al., 2005, Mol Immunol 42, 1205-1214. B2M−/− cells were transduced with these vectors. Puromycin-resistant clones were selected and HLA-E surface expression was analyzed by flow cytometry in pluripotent cells expressing these constructs (FIG. 3C).

Further, a specific classical HLA class I allele single chain fusion protein was constructed and expressed in B2M−/− H1 ESCs to create a “semi-universal” donor cell to facilitate compatibility with recipients. For example, in the U.S. the HLA-A0201 allele is present in 48% of Caucasians, 46% of Hispanics, and 24% of African-Americans, all of which should accept HLA-A0201+ stem cells. See www.allelfrequencies.net and Storkus et al., 1989, PNAS USA 86:2361-2364. The HLA-bBA0201 dimeric single chain fusion construct was introduced in B2M−/− H1 ESCs by foamy virus vectors (FIG. 3B) and the expression of the single chain fusion protein was analyzed by flow cytometry (FIG. 3D).

It should be understood that the foregoing disclosure emphasizes certain specific embodiments of the invention and that all modifications or alternatives equivalent thereto are within the spirit and scope of the invention as set forth in the appended claims.

1-43. (canceled)

44. An isolated cell comprising
a. a genetically engineered disruption of an endogenous β2 microglobulin (B2M) gene,
b. one or more polynucleotides capable of encoding a single chain fusion non-classical human leukocyte antigen (HLA) class I protein, wherein the single chain fusion non-classical HLA class I protein comprises at least a portion of B2M covalently linked to at least a portion of a HLA-F class I α chain,
wherein the single chain fusion classical HLA class I protein is capable of normal function with respect to engaging an inhibitory receptor on the surface of natural killer (NK) cells.

45. An isolated cell comprising
a. a genetically engineered disruption of an endogenous β2 microglobulin (B2M) gene,
b. one or more polynucleotides capable of encoding a single chain fusion non-classical human leukocyte antigen (HLA) class I protein, wherein the single chain fusion non-classical HLA class I protein comprises at least a portion of B2M covalently linked to at least a portion of a HLA-G class I α chain,
wherein the single chain fusion classical HLA class I protein is capable of normal function with respect to engaging an inhibitory receptor on the surface of natural killer (NK) cells.

46. An isolated cell comprising
a. a genetically engineered disruption of an endogenous β2 microglobulin (B2M) gene,
b. one or more polynucleotides capable of encoding a single chain fusion non-classical human leukocyte antigen (HLA) class I protein, wherein the single chain fusion non-classical HLA class I protein comprises at least a portion of B2M covalently linked to at least a portion of a HLA-E class I α chain,
wherein the single chain fusion classical HLA class I protein is capable of normal function with respect to engaging an inhibitory receptor on the surface of natural killer (NK) cells.

47. An isolated cell comprising
a. a genetically engineered disruption of an endogenous β2 microglobulin (B2M) gene,
b. one or more polynucleotides capable of encoding a single chain fusion human leukocyte antigen (HLA) class I protein, wherein the single chain fusion HLA class I protein comprises at least a portion of B2M covalently linked to at least a portion of a HLA-A class I α chain,
wherein the single chain fusion classical HLA class I protein is capable of normal function with respect to engaging an inhibitory receptor on the surface of natural killer (NK) cells.

48. An isolated cell comprising
a. a genetically engineered disruption of an endogenous β2 microglobulin (B2M) gene,
b. one or more polynucleotides capable of encoding a single chain fusion human leukocyte antigen (HLA) class I protein, wherein the single chain fusion HLA class I protein comprises at least a portion of B2M covalently linked to at least a portion of a HLA-B class I α chain,
wherein the single chain fusion classical HLA class I protein is capable of normal function with respect to engaging an inhibitory receptor on the surface of natural killer (NK) cells.

49. An isolated cell comprising
a. a genetically engineered disruption of an endogenous β2 microglobulin (B2M) gene,
b. one or more polynucleotides capable of encoding a single chain fusion human leukocyte antigen (HLA) class I protein, wherein the single chain fusion HLA class I protein comprises at least a portion of B2M covalently linked to at least a portion of a HLA-C class I α chain,
wherein the single chain fusion classical HLA class I protein is capable of normal function with respect to engaging an inhibitory receptor on the surface of natural killer (NK) cells.

50. The cell of claim 44, wherein the cell comprises genetically engineered disruptions in all copies of the B2M gene.

51. The cells of claim 44, wherein the cell further comprises a peptide that is presented by the single chain fusion non-classical HLA class I protein on the cell surface.

52. The cell of any one of claim 51, wherein the peptide is covalently linked to the single chain non-classical HLA class I protein.

53. The cell of claim 44, wherein the B2M protein is a full-length B2M protein.

54. The cell of claim 44, wherein the B2M protein and the non-classical HLA class-I α chain are linked via a linker sequence.

55. The cell of claim 44, wherein the B2M protein and non-classical HLA class-I α chain are linked via a peptide.

56. The cell of claim 44, wherein the B2M protein or the HLA class-I α chain of the single chain fusion non-classical HLA class I protein lacks a leader sequence

57. The cell of claim 44, wherein the cell further comprises one or more recombinant genes capable of encoding a suicide gene product.

58. The cell of claim 57, wherein the suicide gene product comprises a protein selected from the group consisting of thymidine kinase and an apoptotic signaling protein.

59. The cell of claim 44, wherein the cell has a normal karyotype.

60. The cell of claim 44, wherein the cell is a non-transformed cell.

61. The cell of claim 44, wherein the cell is a stem cell.

62. The cell of claim 61, wherein the stem cell is selected from the group consisting of a pluripotent stem cell, a hematopoietic stem cell, an embryonic stem cell, an induced pluripotent stem cell, an adult stem cell, a liver stem cell, a neural stem cell, a pancreatic stem cell and a mesenchymal stem cell.

63. The cell of claim 44, wherein the cell is a differentiated cell.

64. The cell of claim 63, wherein the differentiated cell is selected from the group consisting of a dendritic cell, a pancreatic islet cell, a liver cell, a muscle cell, a keratinocyte, a neuronal cell, a hematopoietic cell, a lymphocyte, a NK cell, a red blood cell, a platelet, a skeletal muscle cell, an ocular cell, a mesenchymal cell, a fibroblast, a lung cell, a GI tract cell, a vascular cell, an endocrine cell, an adipocyte, a marrow stromal cell, an osteoblast, a chrondrocyte, and a cardiomyocyte.

65. The cell of claim 44, wherein the cell is a human cell.

66. A pharmaceutical composition comprising the cell of claim 64 and a physiological compatible buffer.

67. A kit comprising the cell of claim 64.
